(19) United States
(12) Patent Application Publication
(10) Pub. No.: US 2023/0323299 A1
(43) Pub. Date: Oct. 12, 2023

(54) POPULATION OF TREG CELLS FUNCTIONALLY COMMITTED TO EXERT A REGULATORY ACTIVITY AND THEIR USE FOR ADOPTIVE THERAPY

(71) Applicants: INSERM (INSTITUT NATIONAL DE LA SANTÉ ET DE LA RECHERCH MÉDICALE), Paris (FR); MEDECIN ET INNOVATION, Paris (FR); UNIVERSITÉ PARIS CITÉ, Paris (FR)

(72) Inventors: Hélène LE BUANEC, PARIS (FR); Daniel ZAGURY, Paris (FR)

(21) Appl. No.: 18/040,363

(22) PCT Filed: Aug. 2, 2021
(86) PCT No.: PCT/EP2021/071571

§ 371 Date: Feb. 2, 2023

(30) Foreign Application Priority Data
Aug. 3, 2020 (EP) 20305896.1

(51) Int. Cl. C12N 5/0783

(52) U.S. Cl. CPC C12N 5/0637; C12N 2501/01; C12N 2501/15; C12N 2501/2302

(57) ABSTRACT

Natural Treg (nTreg) can potentially suppress cell immune response. Consequently, these CD4+ CD25+ CD127low Foxp3+ T cells are used in adoptive therapy against autoimmune and GVH disease. One difficulty is the varying functional properties depending on the microenvironment that may cause the loss of their suppressive activity and promote TH17- induced inflammatory effects. By ex vivo transdetermination of CD31 TH0 cells, the inventors established and expanded a Foxp3 regulatory T cell population (CD31d-Treg cells) functionally committed to exert a permanent Ag-specific regulatory activity whichever the microenvironmental conditions are. By contrast to nTreg cells, CD31d-Treg cells do not express the IL1 Receptor whose activation is required for IL-17 production. Accordingly, the present invention relates to a population of CD31d-Treg cells functionally committed to exert a regulatory activity and their use for Treg-based adoptive therapy.

### FIELD OF THE INVENTION

The present invention is in the field of medicine, in particular immunology.

### BACKGROUND OF THE INVENTION

Regulatory T cells (Treg cells) are able to promote tolerance against self and non-self-antigens, but have been shown to be numerically or functionally defective in several human auto-immune (1-3) and allo-immune diseases, such as solid organ transplant rejection (4) or graft-versus-host disease (5, 6). The adoptive transfer of Treg cells is a promising therapeutic strategy in these settings.

However, the in vitro induction and expansion of human Treg cells for cell therapy purpose is a major challenge for several reasons. First, human Treg cells are rare in the peripheral blood, especially in patients with autoimmune diseases (1-3). Consequently, it may be difficult to generate a clinically relevant cell dose of Treg cells (7). The adoptive transfer of in vitro expanded Treg cells has been used in phase I/II human clinical trials. Such studies have been conducted in the setting of type I diabetes mellitus (8-10), in prevention of graft-versus-host disease after allogeneic stem cell transplantation (11-15), and in the treatment of Crohn’s disease (16). Many other human clinical studies using Treg adoptive cell transfer in the prevention of acute and chronic graft-versus-host disease, or of allogeneic transplant rejection in solid organ transplantation, are currently ongoing (Table 1). To date, most published data come from uncontrolled, open-label phase I/II studies on small numbers of patients. These studies have confirmed the tolerability of nTreg cell therapy but, designed for safety assessment, do not allow to draw firm conclusions regarding efficacy of Treg adoptive cell transfer. Furthermore, most of these trials used polyclonal but not Ag specific expansion of Treg cells (9, 10, 14, 17) and as such are likely to require for efficacy a much larger amount of regulatory T cells than those administered originating from fresh Peripheral Blood (PB) or Cord Blood (CB) Treg cells. Along this line, first Bluestone et al (10) reported that polyclonally activated Treg cells adoptively transferred at a dose up 2,6. 10⁹ cells to patients suffering from type 1 diabetes although safe, had no beneficial therapeutic effect and secondly several experimental studies in mice have shown that Ag specific Treg cells are more effective than polyclonal Treg cells in inhibiting auto-immune (18-21) and allo-immune reactions (22, 23). Another concern of nTreg-based therapies is the nTreg cell functional instability, given that these cells may functionally behave as pro-inflammatory TH-17-like cells under appropriate microenvironment comprised of IL-1β, IL-6 and IL-2. In effect, Treg cells functional stability may be compromised in inflammatory conditions (24-26). These data prompted us to explore whether ex vivo expanded Ag specific Foxp3 Treg cells generated from TH0 cells (Schiavon) could overcome the above limitations.

Therefore, there is a need for methods providing a new and reliable in vitro protocol allowing the generation and expansion of Ag specific human Foxp3 Treg cells from naive CD4⁺ T cells that will overcome the above limitations.

### SUMMARY OF THE INVENTION

As defined by the claims, the present invention relates to a population of CD31d-Treg cells functionally committed to exert a regulatory activity and their use in particular for Treg-based adoptive therapy.

### DETAILED DESCRIPTION OF THE INVENTION

Natural Treg (nTreg) can potentially suppress cell immune response. Consequently, these CD4+ CD25+ CD127low Foxp3+ T cells are used in adoptive therapy against autoimmune and GVH disease. One difficulty is the varying functional properties depending on the microenvironment that may cause the loss of their suppressive activity and promote TH17-induced inflammatory effects. By ex vivo transdetermination of CD31 TH0 cells, the inventors established and expanded a Foxp3 regulatory T cell population (“CD31d-Treg cells” or “CD31-derived Tregs”) functionally committed to exert a permanent Ag-specific regulatory activity whichever the microenvironmental conditions are. By contrast to nTreg cells, CD31d-Treg cells do not express the IL-1 receptor whose activation is required for IL-17 production.

### Main Definitions

As used herein, the term “T cell” has its general meaning in the art and refers to a type of lymphocytes that play an important role in cell-mediated immunity and are distinguished from other lymphocytes, such as B cells, by the presence of a T-cell receptor (TCR) on the cell surface. In particular, T cells are characterised by the expression of CD3. The term “CD3” refers to the protein complex associated with the T cell receptor is composed of four distinct chains. In mammals, the complex contains a CD3γ chain, a CD3δ chain, and two CD3ε chains. These chains associate with the TCR and the ζ-chain (zeta-chain) to generate an activation signal in T lymphocytes. The TCR, ζ-chain, and CD3 molecules together constitute the TCR complex.

As used herein, the term “CD4” has its general meaning in the art and refers to the T-cell surface glycoprotein CD4. CD4 is a co-receptor of the T cell receptor (TCR) and assists the latter in communicating with antigen-presenting cells. The TCR complex and CD4 each bind to distinct regions of the antigen-presenting MHCII molecule - α1/β1 and β2, respectively.

As used herein, the term “CD4+ T cells” has its general meaning in the art and refers to a subset of T cells which express CD4 on their surface. CD4+ T cells are T helper cells, which either orchestrate the activation of macrophages and CD8+ T cells (Th-1 cells), the production of antibodies by B cells (Th-2 cells) or which have been thought to play an essential role in autoimmune diseases (Th-17 cells).

As used herein, the term “naïve CD4+ T cells” refers to a population of CD4+ T cells characterized by CD45RA⁺CD25⁻CD127⁺.

As used herein, the term “Treg cells” or “regulatory T cells” refers to cells functionally committed, i.e. capable of suppressive activity (i.e. inhibiting proliferation of conventional T cells), either by cell-cell contact or v secretion of inhibitory cytokines such as IL-10. Treg cells include nTreg cells and iTreg cells. As used herein, the term “nTreg cells” or “natural regulatory T cells” has its general meaning in the art and refers to regulatory T cells characterized by their thymic development origin, their CD4⁺CD25⁺CD127⁻Foxp3+ phenotype and their TSDR (Treg specific demethylated region). nTreg cells are thus characterized by the expression of Foxp3 and CD4. Although “iTregs” is commonly used interchangeably with “adaptive Tregs,” the former is perhaps a better nomenclature for all extrathymically derived CD4+ Treg cells. In this context, iTreg cells range from Tr1 cells, which are induced by IL-10, and secrete both IL-10 and TGF-β, to TGF-β-producing Th3 cells (induced by oral antigen tolerizing conditions), to peripheral naïve CD4+CD25-Foxp3-cells that become converted to Foxp3-expressing cells.

As used herein the term “CAR-T cell” refers to a T lymphocyte that has been genetically engineered to express a chimeric antigen receptor (CAR). The term “chimeric antigen receptors (CARs),” as used herein, may refer to artificial T-cell receptors T-bodies, single-chain immunoreceptors, chimeric T-cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In some embodiments, CARs comprise an intracellular activation domain, a transmembrane domain, and an extracellular domain that may vary in length and comprises an antigen binding region. In particular aspects, CARs comprise fusions of single-chain variable fragments (scFv) derived from monoclonal antibodies, fused to CD3-zeta a transmembrane domain and endodomain. In some embodiments, CARs comprise domains for additional co-stimulatory signaling, such as CD3-zeta, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

As used, the term “Foxp3” has its general meaning in the art and refers to a transcriptional regulator which is crucial for the development and inhibitory function of Treg. Foxp3 plays an essential role in maintaining homeostasis of the immune system by allowing the acquisition of full suppressive function and stability of the Treg lineage, and by directly modulating the expansion and function of conventional T-cells. Foxp3 can act either as a transcriptional repressor or a transcriptional activator depending on its interactions with other transcription factors, histone acetylases and deacetylases. Foxp3 inhibits cytokine production and T-cell effector function by repressing the activity of two key transcription factors, RELA and NFATC2. The factor also meediates transcriptional repression of IL2 via its association with histone acetylase KAT5 and histone deacetylase HDAC7. Foxp3 can activate the expression of TNFRSF 18, IL2RA and CTLA4 and repress the expression of IL2 and IFNG via its association with transcription factor RUNX1. Foxp3 inhibits the differentiation of IL17 producing helper T-cells (Th17) by antagonizing RORC function, leading to down-regulation of IL17 expression, favoring Treg development.

As used herein, the term “CD25” has its general meaning in the art and refers to the alpha chain of the human interleukin-2 receptor. The interleukin 2 (IL2) receptor alpha (IL2RA) and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor.

As used herein, the term “CD127” has its general meaning in the art and refers to the interleukin-7 receptor subunit alpha.

As used herein, the term “CD31” has its general meaning in the art and refers to the Platelet endothelial cell adhesion molecule. The tem is also known as PECAM-1, EndoCAM, GPIIA or PECA1.

As used herein, the term “ILIR1” or “CD121a” has its general meaning in the art and refers to the interleukin-1 receptor type 1. ILR1 is the receptor for IL1A, IL1B and IL1RN. After binding to interleukin-1 associates with the coreceptor IL1RAP to form the high affinity interleukin-1 receptor complex which mediates interleukin-1-dependent activation of NF-kappa-B, MAPK and other pathways. Signaling involves the recruitment of adapter molecules such as TOLLIP, MYD88, and IRAK1 or IRAK2 via the respective TIR domains of the receptor/coreceptor subunits. Binds ligands with comparable affinity and binding of antagonist IL1RN prevents association with IL1RAP to form a signaling complex.

As used herein, the term “expression” may refer alternatively to the transcription of a molecule (i.e. expression of the mRNA) or to the translation (i.e. expression of the protein) of a molecule. In some embodiments, detecting the expression may correspond to an intracellular detection. In some embodiments, detecting the expression may correspond to a surface detection, i.e. to the detection of molecule expressed at the cell surface. In some embodiments, detecting the expression may correspond to an extracellular detection, i.e. to the detection of secretion. In some embodiments, detecting the expression may correspond to intracellular, surface and/or extracellular detections. As used herein, the terms “expressing (or +)” and “not expressing (or -)” are well known in the art and refer to the expression level of the phenotypic marker of interest, in that the expression level of the phenotypic marker corresponding to “+” is high or intermediate, also referred as “+/-”. The phenotypic marker corresponding to “-” is a null expression level of the phenotypic marker or also refers to less than 10% of a cell population expressing the said phenotypic marker. In some embodiments, the expression level of cell maker of interest is “low” by comparison with the expression level of that cell marker in the population of cells being analyzed as a whole. More particularly, the term “lo” refers to a distinct population of cells being analyzed as a whole.

As used herein, the term “PBMC” or “peripheral blood mononuclear cells” or “unfractionated PBMC”, as used herein, refers to whole PBMC, i.e. to a population of white blood cells having a round nucleus, which has not been enriched for a given sub-population. Cord blood mononuclear cells are further included in this definition. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer which will preferentially lyse red blood cells.

As used herein, “tolerogenic DCs” refers to DCs capable to induce tolerance. In some embodiments, tolerogenic DCs are capable of secreting more suppressive cytokines such as IL-10 and TGFβ than proinflammatory cytokines such as IL-12, IL-23 or TNFα. In some embodiments, DCs are defined as tolerogenic when they secrete IL-10 and IL-12 in a ratio IL-10: IL-12 > 1.

As used herein, the term “isolated population” refers to a cell population that is removed from its natural environment (such as the peripheral blood or a tissue) and that is isolated, purified or separated, and is at least about 75% free, 80% free, 85% free and preferably about 90%, 95%, 96%, 97%, 98%, 99% free, from other cells with which it is naturally present, but which lack the cell surface markers based on which the cells were isolated.

As used herein, the term “self-peptide antigen” refers to an antigen that is normally expressed in the body from which the regulatory T cells are derived. In some embodiments, self-antigen is comparable to one, or, in some embodiments, indistinct from one normally expressed in a body from which the regulatory T cells are derived, though may not directly correspond to the antigen. In some embodiments, self-antigen refers to an antigen, which when expressed in a body, may result in the education of self-reactive T cells. In some embodiments, self-antigen is expressed in an organ that is the target of an autoimmune disease. In some embodiments, the self-antigen is expressed in a pancreas, thyroid, connective tissue, kidney, lung, digestive system or nervous system. In some embodiments, self-antigen is expressed on pancreatic β cells. Examples of self-peptide antigen, modified self-peptide antigen and over-expressed self-peptide antigen include, but are not limited to, antigenic peptides of insulin, insulin beta, glutamic acid decarboxylase 1 (GAD1), glutamic acid decarboxylase 65 (GAD 65), HSP, thyroglobulin, nuclear proteins, acetylcholine receptor, collagen, thyroid stimulating hormone receptor (TSHR), ICA512(IA-2) and IA-2β (phogrin), carboxypeptidase H, ICA69, ICA12, thyroid peroxidase, native DNA, myelin basic protein, myelin proteolipid protein, acetylchohne receptor components, histocompatibility antigens, antigens involved in graft rejection and altered peptide ligands. Examples of tissue lysate include, but are not limited to, synovial liquid or inflammatory tissue lysate.

As used herein, the term “foreign antigen” refers to a molecule or molecules which is/are not endogenous or native to a mammal which is exposed to it. The foreign antigen may elicit an immune response, e.g. a humoral and/or T cell mediated response in the mammal. Generally, the foreign antigen will result in the production of antibodies there against. Examples of foreign antigens include, but are not limited to, proteins (including a modified protein such as a glycoprotein, a mucoprotein, etc.), nucleic acids, carbohydrates, proteoglycans, lipids, mucin molecules, immunogenic therapeutic agents (including proteins such as antibodies, particularly antibodies comprising non-human amino acid residues, e.g. rodent, chimeric/humanized, and primatized antibodies), toxins (optionally conjugated to a targeting molecule such as an antibody, wherein the targeting molecule may also be immunogenic), gene therapy viral vectors (such as retroviruses and adenoviruses), grafts (including antigenic components of the graft to be transplanted into the heart, lung, liver, pancreas, kidney of graft recipient and neural graft components), infectious agents (such as bacteria and virus or other organism, e.g., protists), alloantigens (i.e. an antigen that occurs in some, but not in other members of the same species) such as differences in blood types, human lymphocyte antigens (HLA), platelet antigens, antigens expressed on transplanted organs, blood components, pregnancy (Rh), and hemophilic factors (e.g. Factor VIII and Factor IX). In some embodiments, the self-peptide antigen or the foreign antigen is soluble.

As used herein, the term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein. By “antibody” or “antibody molecule”, it is intended herein not only whole immunoglobulin molecules but also fragments thereof, such as Fab, F(ab′)2, Fv and other fragments thereof. Similarly, the term antibody includes genetically engineered derivatives of antibodies such as single chain Fv molecules (scFv) and domain antibodies (dAbs). The term “monoclonal antibody” is used herein to encompass any isolated Ab’s such as conventional monoclonal antibody hybridomas, but also to encompass isolated monospecific antibodies produced by any cell, such as for example a sample of identical human immunoglobulins expressed in a mammalian cell line. Suitable monoclonal antibodies which are reactive as described herein may be prepared by known techniques, for example those disclosed in “Monoclonal Antibodies; A manual of techniques”, H Zola (CRC Press, 1988) and in “Monoclonal Hybridoma Antibodies: Techniques and Application”, S G R Hurrell (CRC Press, 1982). The term “antibody” is also used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, Ward et al. (Nature 1989 Oct 12; 341 (6242): 544-6), Holt et al., Trends Biotechnol., 2003, 21(11):484-490; and WO 06/030220, WO 06/003388.

As used herein, the expression “antibody capable of depleting the population of Treg cells that express the IL-1 receptor” refers to any antibody that is able to deplete said populations. As used herein, the term “deplete” with respect to Treg cells that express the IL-1 receptor, refers to a measurable decrease in the number of IL-1R+ Treg cells in the subject. The reduction can be at least about 10%, e.g., at least about 20%, 30%, 40%, 50%, 60%, 70%, 80%, 90%, 95%, 96%, 97%, 98%, 99%, or more. In some embodiments, the term refers to a decrease in the number of IL-1R+ Treg cells in the subject to an amount below detectable limits.

As used herein the term “antibody-dependent cell-mediated cytotoxicity” or “ADCC” refers to a cell-mediated reaction in which non-specific cytotoxic cells (e.g., Natural Killer (NK) cells, neutrophils, and macrophages) recognize bound antibody on a target cell and subsequently cause lysis of the target cell. While not wishing to be limited to any particular mechanism of action, these cytotoxic cells that mediate ADCC generally express Fc receptors (FcRs).

As used herein, the term “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgammal (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

As used herein, the terms “Fc receptor” or “FcR” are used to describe a receptor that binds to the Fc region of an antibody. The primary cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991). To assess ADCC activity of a molecule, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecules of interest may be assessed in vivo, e.g., in an animal model such as that disclosed in Clynes et al., Proc. Natl. Acad. Sci. (USA), 95:652-656 (1998). As used herein, the term Effector cells” are leukocytes which express one or more FcRs and perform effector functions. The cells express at least FcγRI, FCγRII, FcγRIII and/or FcγRIV and carry out ADCC effector function. Examples of human leukocytes which mediate ADCC include peripheral blood mononuclear cells (PBMC), natural killer (NK) cells, monocytes, cytotoxic T cells and neutrophils.

As used herein, the term “complement dependent cytotoxicity” or “CDC” refers to the ability of a molecule to initiate complement activation and lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed.

As used herein, the term “antibody-dependent phagocytosis” or “opsonisation” refers to the cell-mediated reaction wherein nonspecific cytotoxic cells that express FcγRs recognize bound antibody on a target cell and subsequently cause phagocytosis of the target cell.

As used herein, the term “subject” or “patient” refers to a mammal, preferably a human. In the present invention, the terms subject and patient may be used with the same meaning. In some embodiments, the subject is awaiting the receipt of, or is receiving medical care or was/is/will be the object of a medical procedure, or is monitored for the development of an autoimmune inflammatory disease. In some embodiments, the subject is an adult (for example a subject above the age of 18). In some embodiments, the subject is a child (for example a subject below the age of 18). In some embodiments, the subject is an elderly human (for example a subject above the age of 60). In some embodiments, the subject is a male. In some embodiments, the subject is a female.

As used herein, the term “autoimmune inflammatory disease” has its general meaning in the art and include arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis such as plaque psoriasis, gutatte psoriasis, pustular psoriasis, psoriasis of the nails, dermatitis including contact dermatitis, chronic contact dermatitis, allergic dermatitis, allergic contact dermatitis, dermatitis herpetiformis, atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn’s disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen’s encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener’s granulomatosis, agranulocytosis, vasculitides, including vasculitis, large vessel vasculitis, polymyalgia rheumatica, giant cell (Takayasu’s) arteritis, medium vessel vasculitis, Kawasaki’s disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison’s disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet’s or Behcet’s disease, Castleman’s syndrome, Goodpasture’s syndrome, Reynaud’s syndrome, Sjogren’s syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter’s disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto’s disease, chronic thyroiditis (Hashimoto’s thyroiditis); subacute thyroiditis, autoimmune thyroid disease, idiopathic hypothyroidism, Grave’s disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan’s syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger’s disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig’s disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt’s syndrome, adrenalitis, gastric atrophy, presenile dementia, demyelinating diseases such as autoimmune demyelinating diseases, diabetic nephropathy, Dressler’s syndrome, alopecia greata, CREST syndrome (calcinosis, Raynaud’s phenomenon, esophageal dysmotility, sclerodactyl, and telangiectasia), male and female autoimmune infertility, mixed connective tissue disease, Chagas’ disease, rheumatic fever, recurrent abortion, farmer’s lung, erythema multiforme, post-cardiotomy syndrome, Cushing’s syndrome, bird-fancier’s lung, allergic granulomatous angiitis, benign lymphocytic angiitis, Alport’s syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter’s syndrome, Caplan’s syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman’s syndrome, Felty’s syndrome, flariasis, cyclitis such as chronic cyclitis, heterochronic cyclitis, iridocyclitis, or Fuch’s cyclitis, Henoch-Schonlein purpura, human immunodeficiency virus (HIV) infection, echovirus infection, cardiomyopathy, Alzheimer’s disease, parvovirus infection, rubella virus infection, post-vaccination syndromes, congenital rubella infection, Epstein-Barr virus infection, mumps, Evan’s syndrome, autoimmune gonadal failure, Sydenham’s chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck’s disease, cryoglobulinemia, Dupuytren’s contracture, endophthalmia phacoanaphylactica, enteritis allergica, erythema nodosum leprosum, idiopathic facial paralysis, chronic fatigue syndrome, febris rheumatica, Hamman-Rich’s disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain’s thyreoiditis, acquired splenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler’s syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton’s syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis. The term also includes autoimmune inflammatory disease secondary to therapeutic treatment, in particular a treatment with an immune checkpoint inhibitor. Typically the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies. Autoimmune inflammatory diseases also include graft-related diseases, in particular, graft versus host disease (GVDH) and Host-Versus-Graft-Disease (HVGD). Typically GVHD is associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “excipient” refers to any and all conventional solvents, dispersion media, fillers, solid carriers, aqueous solutions, coatings, antibacterial and antifungal agents, isotonic and absorption delaying agents and the like. For human administration, preparations should meet sterility, pyrogenicity, general safety and purity standards as required by regulatory offices, such as, for example, FDA Office or EMA.

As used herein, the term “pharmaceutically acceptable” is meant that the ingredients of a pharmaceutical composition are compatible with each other and not deleterious to the subject to which it is administered. Examples of pharmaceutically acceptable excipient include, but are not limited to, water, saline, phosphate buffered saline, dextrose, glycerol, ethanol and the like or combinations thereof.

### The Population of CD31d-Treg of the Present Invention

The first object of the present invention relates to a population of CD31d-Treg cells having the following phenotype CD4⁺CD25⁺ CD121a⁻CD127⁻Foxp3⁺.

According to the present invention the population of CD31d-Treg thus does not express or express in a low level the IL1R1 protein and/or mRNA.

According to the invention, a low level of IL1R1 means that the mRNA of IL1R1 in the population of CD31d-Treg cells according to the invention is expressed 8 times less than a normal cell and means that the protein IL1R1 in the population of CD31d-Treg cells according to the invention is expressed 15 times less than a normal cell.

In some embodiments, the CD31d-Treg cells of the present invention are CAR-T cells.

In some embodiments, the population of CD3 1d-Treg cells of the present invention is isolated.

In some embodiments, the isolated populations of the invention have been frozen and thawed.

In some embodiments, the expression of the phenotypic is assessed by detecting and/or quantifying binding of a ligand to said phenotypic marker.

In some embodiments, said ligand is an antibody specific of said phenotypic marker, and the method of the invention comprises detecting and/or quantifying a complex formed between said antibody and said phenotypic marker.

Typically, the antibodies are conjugated with a label to facilitate the isolation and detection of population of cells of the interest. As used herein, the terms “label” or “tag” refer to a composition capable of producing a detectable signal indicative of the presence of a target, such as, the presence of a specific phenotypic marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650.

In some embodiments, determining the expression level of phenotypic markers is thus conducted by flow cytometry, immunofluorescence or image analysis, for example high content analysis. Typically, the determination of the expression level of phenotypic markers is conducted by flow cytometry. As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors.

In some embodiments, before conducting flow cytometry analysis, cells are fixed and permeabilized, thereby allowing detecting intracellular proteins (e.g. Foxp3). The expression level of the phenotypic marker of interest is typically determined by comparing the Median Fluorescence Intensity (MFI) of the cells from the cell population stained with fluorescently labeled antibody specific for this marker to the fluorescence intensity (FI) of the cells from the same cell population stained with fluorescently labeled antibody with an irrelevant specificity but with the same isotype, the same fluorescent probe and originated from the same specie (referred as Isotype control). The cells from the population stained with fluorescently labeled antibody specific for this marker and that show equivalent MFI or a lower MFI than the cells stained with the isotype controls are not expressing this marker and then are designated (-) or negative. The cells from the population stained with fluorescently labeled antibody specific for this marker and that show a MFI value superior to the cells stained with the isotype controls are expressing this marker and then are designated (+) or positive. In some embodiments, determining the expression level of a phenotypic marker in a cell population comprises determining the percentage of cells of the cell population expressing the phenotypic marker (i.e. cells “+” for the phenotypic marker).

Preferably, said percentage of cells expressing the phenotypic marker is measured by fluorescence activated cell sorting (FACS). As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest.

Accordingly, FACS can be used with the methods described herein to isolate and detect the population of cells of the present invention. FACS typically involves using a flow cytometer capable of simultaneous excitation and detection of multiple fluorophores, such as a BD Biosciences FACSCanto™ flow cytometer, used substantially according to the manufacturer’s instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Laluza....), data input devices, e.g., interface ports, a mouse, a keyboard, etc., fluid handling components, power sources, etc. Typically, the population of cells is contacted with a panel of antibodies specific for the specific phenotypic markers of interest (i.e. CD4, CD25, CD121a, CD127 and Foxp3). The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In some embodiments, fluorescent beads are those contained in TruCount(TM) tubes, available from Becton Dickinson Biosciences, (San Jose, California). Intracellular flow cytometry typically involves the permeabilization and fixation of the cells. Any convenient means of permeabilizing and fixing the cells may be used in practicing the methods. For example permeabilizing agent typically include saponin, methanol, Tween® 20, Triton X-100TM.

In some embodiments, the population of CD31d-Treg cells of the present invention is characterized by the expression of one additional phenotypic marker. In some embodiments, the marker is selected from the group consisting of CD3, CD8, CD5, CD2, CD31, CD103, CD119, CD120a, CD120b, CD122, CD127, CD134, CD14, CD152, CD154, CD178, CD183, CD184, CD19, CD1a, CD210, CD27, CD28, CD3, CD32, CD4, CD44, CD45RO, CD47, CD49d, CD54, CD56, CD62L, CD69, CD7, CD8, CD80, CD83, CD86, CD95, CD97, CD98, CXCR6, GITR, HLA-DR, IFNalphaRII, IL-18Rbeta, KIR-NKAT2, TGFRII, GZMB, GLNY, TBX21, IRF1, IFNG, CXCL9, CXCL10, CXCR3, CXCR6, IL-18, IL-18Rbeta, Fractalkine, IL-23, IL-31, IL-15, IL-7, MIG, Perforin, TCRalpha/beta, TCRgamma/delta, LAT, ZAP70, CCR5, and CR7. In some embodiments, the additional phenotypic marker is selected from the group consisting of ACE, ACTB, AGTR1, AGTR2, APC, APOA1, ARF1, AXIN1, BAX, BCL2, BCL2L1, CXCR5, BMP2, BRCA1, BTLA, C3, CASP3, CASP9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL5, CCL7, CCL8, CCNB1, CCND1, CCNE1, CCR1, CCR10, CCR2, CCR3, CCR4, CCR5, CCR6, CCR7, CCR8, CCR9, CCRL2, CD154, CD19, CD1a, CD2, CD226, CD244, PDCD1LG1, CD28, CD34, CD36, CD38, CD3E, CD3G, CD3Z, CD4, CD40LG, CD5, CD54, CD6, CD68, CD69, CLIP, CD80, CD83, SLAMF5, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNNB1, CTSC, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR3, CXCR4, CXCR6, CYP1A2, CYP7A1, DCC, DCN, DEFA6, DICER1, DKK1, Dok-1, Dok-2, DOK6, DVL1, E2F4, EBI3, ECE1, ECGF1, EDN1, EGF, EGFR, EIF4E, CD105, ENPEP, ERBB2, EREG, FCGR3A,, CGR3B, FN1, FOXP3, FYN, FZD1, GAPD, GLI2, GNLY, GOLPH4, GRB2, GSK3B, GSTP1, GUSB, GZMA, GZMB, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, ID1, ifna1, ifna17, ifna2, ifna5, ifna6, ifna8, IFNAR1, IFNAR2, IFNG, IFNGR1, IFNGR2, IGF1, IHH, IKBKB, IL10, IL12A, IL12B, IL12RB1, IL12RB2, IL13, IL13RA2, IL15, IL15RA, IL17, IL17R, IL17RB, IL18, IL1A, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin, alpha E (antigen CD103, human mucosal lymphocyte, antigen 1; alpha polypeptide),Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7,MAF, MAP2K1, MDM2, MICA, MICB, MKI67, MMP12, MMP9, MTA1, MTSS1, MYC, MYD88, MYH6, NCAM1, NFATC1, NKG7, NLK, NOS2A, P2X7, PDCD1, PECAM-,, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO, RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STAT6, STK36, TAP1, TAP2, TBX21, TCF7, TERT, TFRC, TGFA, TGFB1, TGFBR1, TGFBR2, TIMP3, TLR1, TLR10, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2. In some embodiments, the additional phenotypic marker is an immune checkpoint protein selected from the group consisting of CD40 (CD40 molecule, TNF receptor superfamily member 5), CD274 (CD274 molecule, also known as B7-H; B7H1; PDL1; PD-L1; PDCDILI, PDCD1LG1), ICOS(inducible T-cell co-stimulator), TNFRSF9 (tumor necrosis factor receptor superfamily member 9, also known as ILA; 4-1BB; CD137; CDw137), TNFRSF18 (tumor necrosis factor receptor superfamily member 18, also known as AITR; GITR; CD357; GITR-D), LAG3(lymphocyte-activation gene 3), HAVCR2 (hepatitis A virus cellular receptor 2), TNFRSF4 (tumor necrosis factor receptor superfamily member 4), CD276(CD276 molecule), CTLA-4 (cytotoxic T-lymphocyte-associated protein 4), PDCD1LG2 (programmed cell death 1 ligand 2, also known as B7DC; Btdc; PDL2; CD273; PD-L2; PDCD1L2; bA574F11.2), VTCN1 (V-set domain containing T cell activation inhibitor 1, also known as B7H4), PDCD1 (programmed cell death 1, also known as PD1; PD-1; CD279; SLEB2; hPD-1; hPD-l; hSLE1), BTLA (B and T lymphocyte associated), CD28 (CD28 molecule), TIGIT (T cell immunoreceptor with Ig and ITIM domains), C10orf54 (chromosome 10 open reading frame 54) and CD27 (CD27 molecule).

### Methods for Generating and Expanding the Population of CD31d-Treg Cells of the Present Invention

A further object of the present invention relates to a method of generating the population of CD31d-Treg cells of the present invention comprising the step of stimulating naïve CD31 + T cells with antigen-pulsed tolerogenic DC (tolDC) in presence of the Treg polarizing medium comprising the combination of IL-2, a cAMP activator, a TGFβ pathway activator, and mTOR inhibitor.

In some embodiments, the naïve CD31+ T cells are obtained by any technique well known in the art from a blood sample. In some embodiments, the naïve CD31+ T cells, are isolated from PBMCs (peripheral blood mononuclear cells) by flow cytometry or by negative selection using a MACS system for example.

In some embodiments, tolerogenic DCs express on their surface the major histocompatibility (MHC) class Ia and/or MHC class Ib. The MHC class Ia presentation refers to the “classical” presentation through HLA-A, HLA-B and/or HLA-C molecules whereas the MHC class Ib presentation refers to the “non-classical” antigen presentation through HLA-E, HLA-F, HLA-G and/or HLA-H molecules.

In some embodiments, tolerogenic DCs express 50% of MHC class Ia molecules and 50% of MHC class Ib molecules on their surface. In some embodiments, tolerogenic DCs express 45% of MHC class Ia molecules and 55% of MHC class Ib molecules on their surface. In some embodiments, tolerogenic DCs express 40% of MHC class Ia molecules and 60% of MHC class Ib molecules on their surface. In some embodiments, tolerogenic DCs express 35% of MHC class Ia molecules and 65% of MHC class Ib molecules on their surface. In some embodiments, tolerogenic DCs express 30% of MHC class Ia molecules and 70% of MHC class Ib molecules on their surface. In some embodiments, tolerogenic DCs express 25% of MHC class Ia molecules and 75% of MHC class Ib molecules on their surface. In some embodiments, tolerogenic DCs express 20% of MHC class Ia molecules and 80% of MHC class Ib molecules on their surface. In some embodiments, tolerogenic DCs express 15% of MHC class Ia molecules and 85% of MHC class Ib molecules on their surface. In some embodiments, tolerogenic DCs express 10% of MHC class Ia molecules and 90% of MHC class Ib molecules on their surface. In some embodiments, tolerogenic DCs express 5% of MHC class Ia molecules and 95% of MHC class Ib molecules on their surface. In some embodiments, tolerogenic DCs express only MHC class Ib molecules on their surface.

In some embodiments, tolerogenic DCs express 50% of HLA-A, HLA-B and/or HLA-C molecules and 50% of HLA-E molecules on their surface. In some embodiments, tolerogenic DCs express 45% of HLA-A, HLA-B and/or HLA-C molecules and 55% of HLA-E molecules on their surface. In some embodiments, tolerogenic DCs express 40% of HLA-A, HLA-B and/or HLA-C molecules and 60% of HLA-E molecules on their surface. In some embodiments, tolerogenic DCs express 35% of HLA-A, HLA-B and/or HLA-C molecules and 65% of HLA-E molecules on their surface. In some embodiments, tolerogenic DCs express 30% of HLA-A, HLA-B and/or HLA-C molecules and 70% of HLA-E molecules on their surface. In some embodiments, tolerogenic DCs express 25% of HLA-A, HLA-B and/or HLA-C molecules and 75% of HLA-E molecules on their surface. In some embodiments, tolerogenic DCs express 20% of HLA-A, HLA-B and/or HLA-C molecules and 80% of HLA-E molecules on their surface. In some embodiments, tolerogenic DCs express 15% of HLA-A, HLA-B and/or HLA-C molecules and 85% of HLA-E molecules on their surface. In some embodiments, tolerogenic DCs express 10% of HLA-A, HLA-B and/or HLA-C molecules and 90% of HLA-E molecules on their surface. In some embodiments, tolerogenic DCs express 5% of HLA-A, HLA-B and/or HLA-C molecules and 95% of HLA-E molecules on their surface. In some embodiments, tolerogenic DCs express only HLA-E molecules on their surface.

Methods for obtaining tolerogenic DCs are well-known in the art. An exemplary method is the generation of tolerogenic DCs from CD14⁺ monocytes. For example, CD14⁺ monocytes are cultured in the presence of GM-CSF and IL-4, or in the presence of GM-CSF and IFNα, for the generation of immature DCs.

Methods for inhibiting MHC class Ia molecules expression or inducing the expression of HLA-E molecules on the surface of tolerogenic DCs are well-known. For instance, the inhibition of the TAP transporter (transporter associated with antigen processing) leads to a decreased expression of MHC class Ia molecules thereby promoting HLA-E molecules expression on the surface of tolerogenic DCs. Exemplary methods to inhibit the TAP transporter in the endoplasmic reticulum include, but are not limited to, CRISPR-CAS-9 technology, silencing RNA, transfected DCs with the UL-10 viral protein from the CMV (cytomegalovirus) or the use of viral proteins. Examples of viral proteins able to inhibit the TAP transporter include, but are not limited to, HSV-1 ICP47 protein, varicella-virus UL49.5 protein, cytomegalovirus US6 protein or gammaherpesvirus EBV BNLF2a protein. Another method is the use of a chemical product to inhibit the expression of MHC class Ia molecules without changing HLA-E expression on the surface of tolerogenic DCs. Examples of chemical products include, but are not limited to, 5′- methyl-5′- thioadenosine or leptomycin B.

The tolerogenic DCs are pulsed in the presence of at least one self-peptide antigen, modified self-peptide antigen, over-expressed self-peptide antigen or foreign antigen.

In some embodiments, IL-2 is used at a concentration ranging from 10 IU/ml to 1000 IU/ml. Within the scope of the invention, the expression “from 10 IU/ml to 1000 IU/ml” includes, without limitation, 15 IU/ml, 20 IU/ml, 25 IU/ml, 30 IU/ml, 35 IU/ml, 40 IU/ml, 45 IU/ml, 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 850 IU/ml, 900 IU/ml, 950 IU/ml. In some embodiments, IL-2 is used at a concentration ranging from 50 IU/ml to 250 IU/ml.

In some embodiments, the cAMP activator added in the culture allows the activation of the cAMP pathway. Examples of cAMP activator include, but are not limited to PGE2 (prostaglandin E2), an EP2 or EP4 agonist, a membrane adenine cyclase activator such as forskolin, or metabotropic glutamate receptors agonists. Examples of PGE2 include, but are not limited to, PGE2 of ref P5640 or P0409 (Sigma-Aldrich), PGE2 of ref 2296 (R&D Systems), PGE2 of ref 2268 (BioVision), PGE2 of ref 72192 (Stemcell), PGE2 of ref ab144539 (Abcam), and PGE2 of ref 14010 (Cayman Chemical). In some embodiments, the cAMP activator, preferably PGE2 is used at a concentration ranging from 0.01 µM to 10 µM. Within the scope of the invention, the expression “from 0.01 µM to 10 µM” includes, without limitation, 0.02 µM, 0.03 µM, 0.04 µM, 0.05 µM, 0.06 µM, 0.07 µM, 0.08 µM, 0.09 µM, 0.1 µM, 0.2 µM, 0.3 µM, 0.4 µM, 0.5 µM, 0.6 µM, 0.7 µM, 0.8 µM, 0.9 µM, 1 µM, 1.5 µM, 2 µM, 2.5 µM, 3 µM, 3.5 µM, 4 µM, 4.5 µM, 5 µM, 6 µM, 7 µM, 8 µM, 9 µM. In some embodiments, PGE2 is at a concentration ranging from 0.03 µM to 1.5 µM.

In some embodiments, the TGFβ pathway activator added in the culture allows the activation of the TGFβ pathway. Examples of TGFβ pathway activators include, but are not limited to, TGFβ family (TGFβ1, TGFβ2, TGFβ3), bone morphogenetic proteins (BMPs), growth and differentiation factors (GDFs), anti-müllerian hormone (AMH), activin, and nodal. Examples of TGFβ include, but are not limited to, TGFβ1 of ref T7039 (Sigma-Aldrich), TGFβ2 of ref T2815 (Sigma-Aldrich), TGFβ3 of ref T5425 (Sigma-Aldrich), human TGFβ1 of ref P01137 (R&D system), human TGFβ1 of ref 580702 (Biolegend), TGFβ1 of ref HZ-1011 (HumanZyme), human TGFβ1 of ref 14-8348-62 (Affymetrix eBioscience). In some embodiments, the pathway activator is used at a concentration ranging from 1 ng/ml to 20 ng/ml. Within the scope of the invention, the expression “from 1 ng/ml to 20 ng/ml” includes, without limitation, 2 ng/ml, 2.5 ng/ml, 3 ng/ml, 3.5 ng/ml, 4 ng/ml, 4.5 ng/ml, 5 ng/ml, 5.5 ng/ml, 6 ng/ml, 6.5 ng/ml, 7 ng/ml, 7.5 ng/ml, 8 ng/ml, 8.5 ng/ml, 9 ng/ml, 9.5 ng/ml, 10 ng/ml, 11 ng/ml, 12 ng/ml, 13 ng/ml, 14 ng/ml, 15 ng/ml, 16 ng/ml, 17 ng/ml, 18 ng/ml, 19 ng/ml. In some embodiments, TGFβ is at a concentration ranging from 2.5 ng/ml to 7.5 ng/ml.

In some embodiments, the mTOR inhibitor added in the culture allows the inhibition of the mTOR pathway. Examples of mTOR inhibitor include, but are not limited to, rapamycin (also named sirolimus) and its analogs (termed rapalogs); wortmannin; theophylline; caffeine; epigallocatechin gallate (EGCG); curcumin; resveratrol; genistein; 3, 3-diindolylmethane (DIM); LY294002 (2-(4-morpholinyl)-8-phenyl-4H-1-benzopyran-4-one); PP242; PP30; Torin1; Ku-0063794; WAY-600; WYE-687; WYE-354; and mTOR and PI3K dual-specificity inhibitors such as GNE477, NVP-BEZ235, PI-103, XL765 and WJD008. Examples of rapamycin include, but are not limited to, rapamycin of ref R0395 (Sigma-Aldrich), rapamycin of ref S1039 (Selleckchem), rapamycin of ref 1292 (Tocris), rapamycin of ref R-5000 (LC Laboratories), rapamycin of ref tlrl-rap (InvivoGen), rapamycin of ref ab 120224 (Abcam), rapamycin of ref R0395 (Sigma-Aldrich). Examples of compounds of the same chemical class than rapamycin used clinically include, but are not limited to, Everolimus (code name RAD001), Temsirolimus (code name CCI-779, NSC 683864), Zotarolimus (code name ABT-578). In some embodiments, the mTOR inhibitor, preferably rapamycin, is used at a concentration ranging from 0.1 nM to 50 nM. Within the scope of the invention, the expression “from 0.1 nM to 50 nM” includes, without limitation, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM.

In some embodiments, the culture medium used in the culture of the invention comprises (i) one or more pH buffering system(s); (ii) inorganic salt(s); (iii) trace element(s); (iv) free amino acid(s); (v) vitamin(s); (vi) hormone(s); (vii) carbon/energy source(s). Examples of inorganic salts include, but are not limited to, calcium bromide, calcium chloride, calcium phosphate, calcium nitrate, calcium nitrite, calcium sulphate, magnesium bromide, magnesium chloride, magnesium sulphate, potassium bicarbonate, potassium bromide, potassium chloride, potassium dihydrogen phosphate, potassium disulphate, di- potassium hydrogen phosphate, potassium nitrate, potassium nitrite, potassium sulphite, potassium sulphate, sodium bicarbonate, sodium bromide, sodium chloride, sodium disulphate, sodium hydrogen carbonate, sodium dihydrogen phosphate, di-sodium hydrogen phosphate, sodium sulphate and a mix thereof. Examples of trace elements include, but are not limited to, cobalt (Co), copper (Cu), iron (Fe), magnesium (Mg), manganese (Mn), molybdenum (Mo), nickel (Ni), selenium (Se), zinc (Zn) and the salts thereof. Examples of free amino acids include, but are not limited to, L-alanine, L-arginine, L-asparagine, L-aspartic acid, L-cysteine, L-cystine, L-glutamine, L-glutamic acid, glycine, L-histidine, L-isoleucine, L-leucine, L-lysine, L-methionine, L-phenylalanine, L-proline, L-serine, taurine, L-threonine, L-tryptophan, L-tyrosine, L-valine and a mix thereof. Examples of vitamins include, but are not limited to, biotin (vitamin H); D-calcium-pantothenate; choline chloride; folic acid (vitamin B9); myo-inositol; nicotinamide; pyridoxal (vitamin B6); riboflavin (vitamin B2); thiamine (vitamin B1); cobalamin (vitamin B12); acid ascorbic; α-tocopherol (vitamin E) and a mix thereof. Examples of carbon/energy sources include, but are not limited to, D-glucose; pyruvate; lactate; ATP; creatine; creatine phosphate; and a mix thereof.

In some embodiments, the culture medium is a commercially available cell culture medium, in particular selected in a group comprising the IMDM (Iscove’s Modified Dulbecco’s Medium) from GIBCO® or the RPMI 1640 medium from GIBCO®. In some embodiments, the culture medium is a serum-free culture medium such as the AIM-V medium from GIBCO®, the X-VIVO 10, 15 and 20 media from LONZA. In some embodiments, the culture medium can be further supplemented with additional compound(s), in particular selected in a group comprising foetal bovine serum, pooled human AB serum, cytokines and growth factors; antibiotic(s), in particular selected in a group comprising penicillin, streptomycin and a mix thereof. In some embodiments, the culture medium is IMDM. In some particular embodiments, the culture medium comprises IMDM cell culture medium; from 1% (w/w) to 5% (w/w) of foetal bovine serum; from 10 IU/ml to 200 IU/ml of penicillin; from 10 IU/ml to 200 IU/ml of streptomycin; from 0.1 mM to 10 mM of a mixture of non-essential amino acids, in particular amino acids selected in a group comprising alanine, asparagine, aspartic acid, cysteine, glutamic acid, glutamine, glycine, proline, serine, and tyrosine; from 0.5 mM to 10 mM of glutamine from 10 mM to 25 mM of HEPES pH 7.6-7.8.

In some embodiments, the culture is performed during at least 5 days, at least 6 days, at least 7 days, at least 8 days. Within the scope of the invention, the expression “at least 5 days” includes, without limitation, 5 days, 6 days, 7 days, 8 days, 9 days, 10 days, 11 days, 12 days, 13 days, 14 days, 15 days.

In some embodiments, a portion of the culture medium is discarded once, twice, three times, four times or five times during the time course of the generation culture and replaced with the same volume of fresh culture medium. Within the scope of the invention the term “portion” is intended to mean at least 20% (v/v), at least 25% (v/v), at least 30% (v/v), at least 35% (v/v), at least 40% (v/v), at least 45% (v/v), at least 50% (v/v), at least 55% (v/v), at least 60% (v/v), at least 65% (v/v), at least 70% (v/v), at least 75% (v/v) of the volume of the culture medium. In some embodiments, 40% (v/v) to 60% (v/v) of the volume of the culture medium of step a) is discarded. In some embodiments, the volume that is discarded is replaced with an identical volume of fresh culture medium.

In some embodiments, the method of the present invention further comprises a step of expanding the population of CD31d-Treg cells of the present invention in the presence of the Treg polarizing medium and in presence of a hypomethylating agent.

As used herein, the term “hypomethylating agent” refers to an agent that reduces or reverses DNA methylation, either at a specific site (e.g., a specific CpG island) or generally throughout a genome. Hypomethylating agents can be referred to as possessing “hypomethylating activity.” By way of example, such activity is measured by determining the methylation state and/or level of a specific DNA molecule or site therein, or the general methylation state of a cell, on parallel samples that have and have not been treated with the hypomethylating agent (or putative hypomethylation agent). A reduction in methylation in the treated (versus the untreated) sample indicates that the agent has hypomethylating activity. Exemplary hypomethylating agents include the following compounds, decitabine (5-aza-deoxycytidine), zebularine, isothiocyanates, azacitidine (5-azacytidine), 5-fluoro-2′ -deoxycytidine, 5,6-dihydro-5-azacytidine, ethionine, S-adenosyl-L-homocysteine, mitoxantrone, neplanocin A, 3-deazaneplanocin A, cycloleucine, hydralazine, phenylhexyl isothiocyanate, curcumin, parthenolide, and SGI-1027.

In some embodiments, the method of the present invention further comprises a step of expanding the population of CD31d-Treg cells of the present invention in the presence of the Treg polarizing medium and in presence of a TCRαβ cell activator.

Examples of TCR αβ activator include, but are not limited to, anti-TCR αβ antibody such as purified anti-human TCR α/β antibody (ref 306702, Biolegend), Anti-Human alpha beta TCR antibody (ref 11-9986-41, eBioscience), anti-human TCR of (ref 563826, BD Biosciences), TCR alpha/beta antibody (ref GTX80083, GeneTex); anti-CD3 antibody such as purified anti-human CD3 antibody (ref 344801, BioLegend), anti-CD3 antibody (ab5690, Abcam), anti-human CD3 purified (ref 14-0038-80, eBioscience), CD3 antibody (ref MA5-17043, Invitrogen antibodies), CD3 monoclonal antibody (ref ALX-804-822-C100, Enzo Life Sciences), human CD3 antibody (ref 130-098-162, Miltenyi Biotec); mitogen such as pokeweed mitogen, ionomycin, phorbol myristate acetate (PMA), phytohaemagglutinin (PHA), lipopolysaccharide (LPS), superantigen such as staphylococcal enterotoxins (SPE), retroviral antigens, streptococcal antigens, mycoplasma antigens, mycobacterium antigens, viral antigens (e.g., a superantigen from mouse mammary tumor virus, rabies virus or herpes virus) and endoparasitic antigens (e.g., protozoan or helminth antigens). In some embodiments, the TCRaβ cell activator is soluble in the culture medium. In some embodiments, the polyclonal TCR αβ cell activator is coated to the culture plate.

In some embodiments, the culture for expanding the population of CD31d-Treg cells of the present invention is performed during at least 5 days, at least 6 days, at least 7 days, at least 8 days. Within the scope of the invention, the expression “at least 5 days” includes, without limitation, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more.

### Uses of the Population of CD31d-Treg Cells of the Present Invention

As disclosed herein, the population of CD3 1d-Treg cells of the present invention remains stable when placed in inflammatory conditions.

As used herein, “inflammatory condition” refers to a medium enriched in aromatic acid, preferably in tryptophan, such as for example IMDM, comprising inflammatory cytokines such as for example IL-1β (10 ng/ml), IL-6 (30 ng/ml), IL-21 (50 ng/ml), IL-23 (30 ng/ml), IL-2 (100 UI/ml). A method for determining if a population of regulatory T cells remains stable in inflammatory condition comprises culturing the regulatory T cells in the inflammatory condition medium as described here above in the presence of anti-CD3 (4 µg/ml), preferably coated, and anti-CD28 (4 µg/ml), preferably in a soluble form. After 36h to 72h of culture, the presence of IL-17 in the culture supernatant is measured. The recognition of IL-17 in the culture supernatant may be carried out by conventional methods known in the art such as, for example, a sandwich ELISA anti-IL-17. Briefly, after coated the plate with a capture anti-IL-17 antibody, the culture supernatant is added to each well with a dilution series. After incubation, a detection anti-IL-17 antibody is added to each well. The ELISA is developed by any colorimetric means known in the art such as, for example, using detection antibody labelled with biotin, a poly-streptavidin HRP amplification system and an o-phenylenediamine dihydrochloride substrate solution. An IL-17 level inferior to 200 ng/ml, 100 ng/ml, 50 ng/ml corresponds to no secretion or low secretion of IL-17.

As used herein, “stable” refers to no secretion or a low secretion of IL-17, i.e. inferior to 200 ng/ml, 100 ng/ml, 50 ng/ml and still capable of suppressive capacity, i.e. inhibiting proliferation of conventional T cells as shown in the Examples.

Thus the population of CD31d-Treg cells of the present invention is thus suitable for the treatment of autoimmune inflammatory diseases.

Thus a further object of the present invention relates to a method of treating an autoimmune inflammatory disease in a subject in need thereof comprising administering a therapeutically effective amount of the population of CD3 1d-Treg cells of the present invention.

Thus the population of CD31d-Treg cells of the present invention can be utilized in methods and compositions for adoptive immunotherapy in accordance with known techniques, or variations thereof that will be apparent to those skilled in the art based on the instant disclosure. See, e.g., U.S. Pat. Application Publication No. 2003/0170238 to Gruenberg et al; see also U.S. Pat. No. 4,690,915 to Rosenberg. Currently, most adoptive immunotherapies are autolymphocyte therapies (ALT) directed to treatments using the patient’s own immune cells. Typically, the treatments are accomplished by removing the patient’s lymphocytes and processing said cells by the method herein disclosed for generating the population of CD31d-Treg of the present invention. Once the Treg cells are prepared, these ex vivo cells are reinfused into the patient to enhance the immune system to induce tolerance. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer’s lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

Thus a further object of the invention is a pharmaceutical composition the population of CD3 1d-Treg cells of the present invention and at least one pharmaceutically acceptable excipient.

In some embodiments, the pharmaceutical composition of the invention comprises, consists essentially of or consists of at least 10⁴, 10⁵, 10⁶, 10⁷, 10⁸, 10⁹, 10¹⁰ of CD3 1d-Treg cells of the present invention as active principle.

The pharmaceutical composition may be produced by those of skill, employing accepted principles of treatment. Such principles are known in the art, and are set forth, for example, in Braunwald et al., eds., Harrison’s Principles of Internal Medicine, 19th Ed., McGraw-Hill publisher, New York, N.Y. (2015), which is incorporated by reference herein. The pharmaceutical composition may be administered by any means that achieve their intended purpose. For example, administration may be by parenteral, subcutaneous, intravenous, intradermal, intramuscular, intraperitoneal, transdermal, or buccal routes. The pharmaceutical compositions may be administered parenterally by bolus injection or by gradual perfusion over time. The pharmaceutical compositions typically comprise suitable pharmaceutically acceptable carriers comprising excipients and auxiliaries which may facilitate processing of the active compounds into preparations which can be used pharmaceutically. The pharmaceutical compositions may contain from about 0.001 to about 99 percent, or from about 0.01 to about 95 percent of active compound(s), together with the excipient.

### Method of Promoting the in Vivo Expansion of a Population of CD31d-Treg Cells According to the Present Invention in a Subject in Need Thereof:

A further object of the present invention relates to a method of promoting the in vivo expansion of a population of CD31d-Treg cells according to the present invention (i.e. CD4⁺CD25⁺ CD121a⁻CD127⁻Foxp3⁺ T cells) in a subject in need thereof comprising administering to the patient a therapeutically effective amount of an antibody capable of depleting the population of Treg cells that express the IL-1 receptor (IL1R1).

A further object of the present invention relates to a method of treating an autoimmune inflammatory disease in a subject in need thereof comprising administering to the patient a therapeutically effective amount of an antibody capable of depleting the population of Treg cells that express the IL-1 receptor.

In some embodiments, the antibody is a chimeric antibody, a humanized antibody or a human antibody.

In some embodiments, the antibody suitable for depletion of IL-1R+ Treg cells mediates antibody-dependent cell-mediated cytotoxicity.

In some embodiments, the antibody suitable for depletion of IL-1R+ Treg cells is a full-length antibody. In some embodiments, the full-length antibody is an IgG 1 antibody. In some embodiments, the full-length antibody is an IgG3 antibody.

In some embodiments, the antibody suitable for depletion of IL-1R+ Treg cells comprises a variant Fc region that has an increased affinity for FcγRIA, FcγRIIA, FcγRIIB, FcγRIIIA, FcγRIIIB, and FcγRIV. In some embodiments, the antibody of the present invention comprises a variant Fc region comprising at least one amino acid substitution, insertion or deletion wherein said at least one amino acid residue substitution, insertion or deletion results in an increased affinity for FcγRIA, FcγRIIA, FcγRIIB, FcγRIIIA, FcγRIIIB, and FcγRIV, In some embodiments, the antibody of the present invention comprises a variant Fc region comprising at least one amino acid substitution, insertion or deletion wherein said at least one amino acid residue is selected from the group consisting of: residue 239, 330, and 332, wherein amino acid residues are numbered following the EU index. In some embodiments, the antibody of the present invention comprises a variant Fc region comprising at least one amino acid substitution wherein said at least one amino acid substitution is selected from the group consisting of: S239D, A330L, A330Y, and 1332E, wherein amino acid residues are numbered following the EU index.

In some embodiments, the glycosylation of the antibody suitable for depletion of IL-1R+ Treg cells is modified. For example, an aglycosylated antibody can be made (i.e., the antibody lacks glycosylation). Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. Additionally or alternatively, an antibody can be made that has an altered type of glycosylation, such as a hypofucosylated or non-fucosylated antibody having reduced amounts of or no fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the present invention to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation or are devoid of fucosyl residues. Therefore, in some embodiments, the human monoclonal antibodies of the present invention may be produced by recombinant expression in a cell line which exhibit hypofucosylation or non-fucosylation pattern, for example, a mammalian cell line with deficient expression of the FUT8 gene encoding fucosyltransferase. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields, R.L. et al, 2002 J. Biol. Chem. 277:26733-26740). PCT Publication WO 99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al, 1999 Nat. Biotech. 17: 176-180). Eureka Therapeutics further describes genetically engineered CHO mammalian cells capable of producing antibodies with altered mammalian glycosylation pattern devoid of fucosyl residues (http://www.eurekainc.com/a&boutus/companyoverview.html). Alternatively, the human monoclonal antibodies of the present invention can be produced in yeasts or filamentous fungi engineered for mammalian- like glycosylation pattern and capable of producing antibodies lacking fucose as glycosylation pattern (see for example EP1297172B1).

In some embodiments, the antibody suitable for depletion of IL-1R+ Treg cells mediates complement dependant cytotoxicity.

In some embodiments, the antibody suitable for depletion of IL-1R+ Treg cells mediates antibody-dependent phagocytosis.

In some embodiments, the antibody suitable for depletion of IL-1R+ Treg cells is a multispecific antibody comprising a first antigen binding site directed against IL-1R and at least one second antigen binding site directed against an effector cell as above described. In said embodiments, the second antigen-binding site is used for recruiting a killing mechanism such as, for example, by binding an antigen on a human effector cell. In some embodiments, an effector cell is capable of inducing ADCC, such as a natural killer cell. For example, monocytes, macrophages, which express FcRs, are involved in specific killing of target cells and presenting antigens to other components of the immune system. In some embodiments, an effector cell may phagocytose a target antigen or target cell. The expression of a particular FcR on an effector cell may be regulated by humoral factors such as cytokines. An effector cell can phagocytose a target antigen or phagocytose or lyse a target cell. Suitable cytotoxic agents and second therapeutic agents are exemplified below, and include toxins (such as radiolabeled peptides), chemotherapeutic agents and prodrugs. In some embodiments, the second binding site binds to a Fc receptor as above defined. In some embodiments, the second binding site binds to a surface molecule of NK cells so that said cells can be activated. In some embodiments, the second binding site binds to NKp46. Exemplary formats for the multispecific antibody molecules of the present invention include, but are not limited to (i) two antibodies cross-linked by chemical heteroconjugation, one with a specificity to a specific surface molecule of ILC and another with a specificity to a second antigen; (ii) a single antibody that comprises two different antigen-binding regions; (iii) a single-chain antibody that comprises two different antigen-binding regions, e.g., two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In : Antibody Engineering, Springer Berlin Heidelberg (2010)); (v) a chemically-linked bispecific (Fab′)2 fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivaient bispecific binding protein consisting of two identical Fab fragments linked to a different Fab fragment; (ix) a so-called Scorpion molecule, comprising, e.g., two scFvs fused to both termini of a human Fab-arm; and (x) a diabody. Another exemplary format for bispecific antibodies is IgG-like molecules with complementary CH3 domains to force heterodimerization. Such molecules can be prepared using known technologies, such as, e.g., those known as Triomab/Quadroma (Trion Pharma/Fresenius Biotech), Knob-into-Hole (Genentech), CrossMAb (Roche) and electrostatically-matched (Amgen), LUZ-Y (Genentech), Strand Exchange Engineered Domain body (SEEDbody)(EMD Serono), Biclonic (Merus) and DuoBody (Genmab A/S) technologies.

In some embodiments, the antibody suitable for depletion of IL-1R+ Treg cells is conjugated to a therapeutic moiety, i.e. a drug. The therapeutic moiety can be, e.g., a cytotoxin, a chemotherapeutic agent, a cytokine, an immunosuppressant, an immune stimulator, a lytic peptide, or a radioisotope. Such conjugates are referred to herein as an “antibody-drug conjugates” or “ADCs”.

In some embodiments, the antibody suitable for depletion of IL-1R+ Treg cells is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxy anthracin dione; a tubulin- inhibitor such as maytansine or an analog or derivative thereof; an antimitotic agent such as monomethyl auristatin E or F or an analog or derivative thereof; dolastatin 10 or 15 or an analogue thereof; irinotecan or an analogue thereof; mitoxantrone; mithramycin; actinomycin D; 1-dehydrotestosterone; a glucocorticoid; procaine; tetracaine; lidocaine; propranolol; puromycin; calicheamicin or an analog or derivative thereof; an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof; an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins such as PAPI, PAPII, and PAP-S, momordica charantia inhibitor, curcin, crotin, sapaonaria officinalis inhibitor, gelonin, mitogellin, restrictocin, phenomycin, and enomycin toxins; ribonuclease (RNase); DNase I, Staphylococcal enterotoxin A; pokeweed antiviral protein; diphtherin toxin; and Pseudomonas endotoxin.

In some embodiments, the antibody suitable for depletion of IL-1R+ Treg cells is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anticancer (US5663149) and antifungal activity (Pettit et al., (1998) Antimicrob. Agents and Chemother. 42: 2961-2965. For example, auristatin E can be reacted with para-acetyl benzoic acid or benzoylvaleric acid to produce AEB and AEVB, respectively. Other typical auristatin derivatives include AFP, MMAF (monomethyl auristatin F), and MMAE (monomethyl auristatin E). Suitable auristatins and auristatin analogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the antibody suitable for depletion of IL-1R+ Treg cells is conjugated to pyrrolo[2,1-c][1,4]- benzodiazepine (PDB) or an analog, derivative or prodrug thereof. Suitable PDBs and PDB derivatives, and related technologies are described in, e.g., Hartley J. A. et al., Cancer Res 2010; 70(17): 6849-6858; Antonow D. et al., Cancer J 2008; 14(3) : 154-169; Howard P.W. et al., Bioorg Med Chem Lett 2009; 19: 6463-6466 and Sagnou et al., Bioorg Med Chem Lett 2000; 10(18) : 2083-2086. In some embodiments, the antibody is conjugated to pyrrolobenzodiazepine (PBD) as typically described in WO2017059289.

In some embodiments, the antibody suitable for depletion of IL-1R+ Treg cells is conjugated to a cytotoxic moiety selected from the group consisting of an anthracycline, maytansine, calicheamicin, duocarmycin, rachelmycin (CC-1065), dolastatin 10, dolastatin 15, irinotecan, monomethyl auristatin E, monomethyl auristatin F, a PDB, or an analog, derivative, or prodrug of any thereof.

In some embodiments, the antibody suitable for depletion of IL-1R+ Treg cells is conjugated to an anthracycline or an analog, derivative or prodrug thereof. In some embodiments, the antibody is conjugated to maytansine or an analog, derivative or prodrug thereof. In some embodiments, the antibody is conjugated to calicheamicin or an analog, derivative or prodrug thereof. In some embodiments, the antibody is conjugated to duocarmycin or an analog, derivative or prodrug thereof. In some embodiments, the antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethyl auristatin E or an analog, derivative or prodrug thereof. In some embodiments, the antibody is conjugated to monomethyl auristatin F or an analog, derivative or prodrug thereof. In some embodiments, the antibody is conjugated to pyrrolo[2,1-c][1,4]-benzodiazepine or an analog, derivative or prodrug thereof. In some embodiments, the antibody is conjugated to irinotecan or an analog, derivative or prodrug thereof.

In some embodiments, the antibody suitable for depletion of IL-1R+ Treg cells is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immunostimulatory nucleic acid (e.g., an immunostimulatory CpG motif-containing DNA molecule). In some embodiments, the antibody is conjugated to an aptamer or a ribozyme.

Techniques for conjugating molecule to antibodies, are well-known in the art (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies ‘84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.) Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J.Y., Bajjuri, K.M., Ritland, M., Hutchins, B.M., Kim, C.H., Kazane, S.A., Halder, R., Forsyth, J.S., Santidrian, A.F., Stafin, K., et al. (2012). Synthesis of site-specific antibody-drug conjugates using unnatural amino acids. Proc. Natl. Acad. Sci. USA 109, 16101-16106.; Junutula, J.R., Flagella, K.M., Graham, R.A., Parsons, K.L., Ha, E., Raab, H., Bhakta, S., Nguyen, T., Dugger, D.L., Li, G., et al. (2010). Engineered thio-trastuzumab-DM1 conjugate with an improved therapeutic index to target humanepidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. Conjugation to unnatural amino acids that have been incorporated into the antibody is also being explored for ADCs; however, the generality of this approach is yet to be established (Axup et al., 2012). In particular the one skilled in the art can also envisage Fc-containing polypeptide engineered with an acyl donor glutamine-containing tag (e.g., Gin-containing peptide tags or Q- tags) or an endogenous glutamine that are made reactive by polypeptide engineering (e.g., via amino acid deletion, insertion, substitution, or mutation on the polypeptide). Then a transglutaminase, can covalently crosslink with an amine donor agent (e.g., a small molecule comprising or attached to a reactive amine) to form a stable and homogenous population of an engineered Fc-containing polypeptide conjugate with the amine donor agent being site- specifically conjugated to the Fc-containing polypeptide through the acyl donor glutamine- containing tag or the accessible/exposed/reactive endogenous glutamine (WO 2012059882).

### Populations of Treg Cells Engineered to Repress the Expression of IL-1R

A further object of the present invention relates to a population of Treg cells engineered to repress the expression of the IL-1 receptor (IL1R1).

In some embodiments, the expression of IL-1R is repressed by using an endo nuclease. In some embodiments, the expression of IL-1R is repressed by using a CRISPR-associated endonuclease. CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each includes a hybridization domain which allows the molecules to associate, e.g., through hybridization. Artificial CRISPR/Cas systems can be generated which inhibit IL-1R, using technology known in the art, e.g., that are described in U.S. Publication No. 20140068797, WO2015/048577, and Cong (2013) Science 339: 819-823. Other artificial CRISPR/Cas systems that are known in the art may also be generated which inhibit IL-1R, e.g., that described in Tsai (2014) Nature Biotechnol., 32:6 569-576, U.S. Pat. Nos. 8,871,445; 8,865,406; 8,795,965; 8,771,945; and 8,697,359, the contents of which are hereby incorporated by reference in their entirety. Such systems can be generated which inhibit IL-1R, by, for example, engineering a CRISPR/Cas system to include a gRNA molecule comprising a targeting sequence that hybridizes to a sequence of the IL-1R gene. In some embodiments, the gRNA comprises a targeting sequence which is fully complementarity to 15-25 nucleotides, e.g., 20 nucleotides, of the IL-1R gene. In some embodiments, the 15-25 nucleotides, e.g., 20 nucleotides, of the IL-1R gene, are disposed immediately 5′ to a protospacer adjacent motif (PAM) sequence recognized by the Cas protein of the CRISPR/Cas system (e.g., where the system comprises a S. pyogenes Cas9 protein, the PAM sequence comprises NGG, where N can be any of A, T, G or C).

In some embodiments, the population of Treg cells is a population of CAR-T cells. Thus in some embodiments, the population of Treg cells is also engineered to express a chimeric antigen receptor (CAR). In some embodiments, the portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

In some embodiments, the invention provides a number of chimeric antigen receptors (CAR) comprising an antigen binding domain (e.g., antibody or antibody fragment, TCR or TCR fragment) engineered for specific binding to an auto-antigen of interest.

In some embodiments, the Treg cell is transduced with a viral vector encoding a CAR. In some embodiments, the viral vector is a retroviral vector. In some embodiments, the viral vector is a lentiviral vector. In some embodiments, the cell may stably express the CAR. In some embodiments, the Treg cell is transfected with a nucleic acid, e.g., mRNA, cDNA, DNA, encoding a CAR.

In some embodiments, the antigen binding domain of a CAR of the invention (e.g., a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

The population of Treg cells obtained by the method herein disclosed may find various applications. More particularly, the population of T cells is suitable for the adoptive immunotherapy. Adoptive immunotherapy is an appropriate treatment for autoimmune inflammatory diseases.

A further object of the present invention relates to a method of treating an autoimmune inflammatory disease in a patient in need thereof comprising administering to the patient a therapeutically effective amount of a population of Treg cells engineered to repress the expression of IL-1R.

The invention will be further illustrated by the following figures and examples. However, these examples and figures should not be interpreted in any way as limiting the scope of the present invention.

### FIGURES

FIG. 1: Frequency of Foxp3+ cells in naive cells isolated from CBMCs and PBMCs upon polyclonal stimulation under tolerogenic medium. Average percentage and range of FOXP3 induction on day 21-stimulated CD4+ CD127+ CD45RA+ CD25- cells (CD4+ TH0) isolated from CBMCs n= (5) or PBMCs n= (6) in presence of IL-2 (100IU/ml) PGE2 (500 nM), TGFβ1 (5 ng/ml) and Rapa (10 nm). Naïve nTreg cells (CD4+ CD127- CD45RA+ CD25+ Foxp3+) stimulated and expanded under the same culture condition were used as control samples (n= 6).

FIG. 2: Naive CD31+ TH0 cells transdetemined more efficiently into Foxp3 regulatory Treg cells than CD31- TH0 cells. (A1) Comparison of CD31 frequency in naive CD4+ T cells isolated from CBMCs and PBMCs. CD4+ T cells from CBMCs (n=7) and PBMCs (n=19) were stained with mAbs specific for CD31, CD127, CD45RA, and CD25 and analyzed by flow cytometry. Gray points indicate the relative frequency of CD31+ T cells among the CD4+ TH0 cells in individual samples. (A2) CD31 frequency in naive CD4+ T cells declined with age. Percentage of CD31+ in CD4+ TH0 evaluated by flow cytometry cells is plotted against donor age. (B) Increased frequency of Foxp3+ cells in CD31+ naive cells isolated from PBMCs compared to their CD31- naïve cells counterparts upon polyclonal stimulation with tolerogenic medium. Flow cytometry analysis of Foxp3 and CD25 expression on day 21 stimulated CD31+ (n=8) or CD31- (n=8) naive T cells in presence of PGE2, TGFβ1 and Rapa. Naive CD4+ T cells isolated from CBMCs (n=5) and naïve nTreg cells (n=6) stimulated in the same condition were used as control. (B1) Representative plots for FOXP3 and CD25 staining (with numbers indicating the percentage of FOXP3+ cells gated on viable cells) and (B2 and B3) Summary Plot indicating the frequence (B2) and the level (B3) of Foxp3 expression (MFI ratio FOXP3+/MFI ratio Foxp3-) mean +/- SEM.

FIG. 3: CD31d-Foxp3 Treg cells suppressive function is governed by their structural characteristics. (A) Ex vivo suppressive capacity of CD31d-Foxp3 Treg cells. The suppressive capacity was evaluated in quiescent (A1) and inflammatory (A2) conditions with the standard polyclonal nTreg assay. CFSE-labeled Tconvs were cocultured with CD31d-Foxp3 Treg cells at different ratios. The percent inhibition of TconvCFSE proliferation is depicted. Fresh nTreg cells served as controls. (A3) IL-17 production by CD31d Foxp3 Treg measured in supernatant culture by ELISA. (B) Downregulation of IL-1R1 signaling pathway in CD31d-Foxp3 Treg cells. Histograms indicating the Il1R1 mRNA (B1) and protein expression (B2) in expanded naive nTreg and CD31d-Foxp3 Treg cells evaluated by qRT-PCR and flow cytometry respectively. pSTAT1 responses in cells stimulated with IL1b for 30 min. Representative dot plot (B3) and Mean ± SEM (B4) of pSTAT1 ratio (MFI at 30 min/MFI at baseline) is shown (n = 3). (C1) Scatterplot indicating the FOXP3-CNS2 and promoter methylation levels of the CD31d-Foxp3 Treg cells and expanded naive nTreg as assessed by bisulfite pyrosequencing. (C2) Foxp3 nuclear localization in CD31d-Foxp3 Treg cells and expanded naive nT depicting in immune fluorescence images 63X taken on slides labeled with anti-FOXP3 (red) antibodies and counterstained with DAPI for the nuclei. Expanded CD4+ CD25- Foxp3- are used as control. **P < 0.01; ***P < 0.001; ****P < 0.0001.

FIG. 4: Generation and biological characteristics of Ag specific CD31d Foxp3 Treg cells. Representative plots showing expression of CD45RA and CD45RO (A1) and CD25 (A2) by naive TH0 cells stimulated for 21 d with unpulsed or OVA-pulsed autologous tol-DC in presence of tolerogenic medium and IL-2. Histograms indicating the percentage of CD45RA (A3) and CD25 (A4) expressed by these stimulated naive TH0 cells (n = 3). (B1) Dot plot depicting CD25 and Foxp3 expression in 14 and 21-day stimulated CD31 naive TH0 cells with OVA-pulsed tol-DC in presence of tolerogenic medium. (B2) Histogram showing expression level of Foxp3 in 21-day stimulated CD31 naive TH0 cells with OVA or LS-pulsed tolDC in presence of tolerogenic medium. (C) Ex vivo suppressive capacity of OVA specific CD31d-Foxp3 Treg cells. The suppressive capacity of CD31d-Foxp3 Treg cells was evaluated in quiescent (C1) and inflammatory (C2) conditions with an antigen specific nTreg assay. CFSE-labeled Tconvs were cocultured with CD31d-Foxp3 Treg cells at different ratios. The percent inhibition of TconvCFSE proliferation is depicted. Fresh nTreg cells served as controls. (C3) IL-17 production by OVA specific CD31d Foxp3 Treg measured in supernatant culture by ELISA.

FIG. 5: Sorting iTreg: Percentage CD4 naïve CD31+. (A) Healthy donor, (B) Patient A: Female, White, 37 years, Pre-Treatment (recently diagnosed), Other symptom/dis: Raynaud’s syndrome. Other medication: Ibuprofen & Robaxin, (C) Patient B: Female, Black, 65 years, treated with Plaquenil, Stable, Other treatments: Omeprazole, Potassium Chloride, Reclast, Symbicort, Taztia XT, Viagra. Other symptoms/dis.: Arthralgias, COPD, Hypertension, Osteoporosis, Osteopenia, Vit D Deficiency.

FIG. 6: iTreg Phenotype Foxp3. (A) Healthy Donor (HD) control and same culture media, and (B) Patient A (upper panel) B (lower panel).

### EXAMPLE 1

### Material & Methods

1) Human blood samples: Peripheral Blood samples were obtained from healthy donors through Etablissement Français du Sang (EFS, Paris, France). Umbilical cord blood (UCB) samples were obtained from normal term deliveries, after maternal informed consent and stored in the cord blood bank according to approved institutional guidelines (Cellular therapy unit, Saint Louis hospital, Paris, Assistance Publique-Hôpitaux de Paris, France). Blood cells were collected using standard procedures. The study was performed according to the Helsinki declaration, and the study protocol was reviewed and approved by the local Ethics Committee. All samples were de-identified prior to use in this study.

2) Cell purification. Peripheral blood mononuclear cells (PBMCs) and UCB were isolated by density gradient centrifugation on Ficoll-Hypaque (Pharmacia, St Quentin en Yvelines, France). PBMCs were used either as fresh cells or stored frozen in liquid nitrogen. CD4+ T cell subsets and T cell-depleted accessory cells (ΔCD3 cells) were isolated from either fresh or frozen PBMCs or UCB. All CD4+ T cells were positively selected with a CD4+ T cell isolation kit (Miltenyi Biotec, Bergisch-Gladbach, Germany), yielding CD4+ T cell populations at a purity of 96-99%. Subsequently, selected CD4+ T cells were labelled with anti-CD25 (B1.49.9)-PC5.5 (Beckman Coulter), anti-CD127 (HIL-7R-M21)-BV421 (BD Biosciences), anti-CD45RA (REA562)-FITC (Miltenyi) and anti-CD31 (WM59)-PE (Biolegend) before being sorted into naive nTreg (CD4+CD127-/lowCD25highCD45RA+) and naive CD31+ or CD31- Tconv (CD4+CD127+CD25neg/ dimCD45RA+CD31+/-) subpopulations using a FACSARIAIII Cell Sorter (Becton Dickinson, Le Pont Claix, France). Postsort analysis confirmed that the purity for each cell type was routinely greater than 90% and that more than 90 % of sorted nTreg expressed FOXP3. T cell-depleted accessory cells (ΔCD3 cells) were isolated by negative selection from PBMCs by incubation with anti-CD3-coated Dynabeads (Dynal Biotech, Oslo, Norway) and were irradiated at 5000 rad (referred to as ΔCD3-feeder).

3) Culture. Immature DCs (iDCs) were generated from MACS-isolated CD14+ human monocytes by 6-day cultivation with 100 ng/mL of GM-CSF and 10 ng/mL of IL-4. Their maturation (mDC) was induced by stimulation with LPS (100 ng/mL, Sigma-Aldrich, St. Louis, MO, USA) for an additional 48 h.

Purified naive nTreg and Tconv cell subsets were cultured separately in IMDM medium containing 2 mM L-glutamine, 100 U/mL penicillin-streptomycin, 1 mM sodium pyruvate and 10% human AB serum, (referred to as complete medium) (Invitrogen, Cergy-Pontoise, France) in 96-well U-bottom plates (Falcon/Becton Dickinson). All cultures were incubated at 37° C. with 5% CO2 and 95% air.

For polyclonal iTreg generation, cells were stimulated with plate-bound anti-human CD3 (OKT3) mAb (eBioscience, San Diego, CA) at the concentration of 1 µg/mL, soluble anti-human CD28 (CD28.2) mAb (Becton Dickinson, 2 µg/mL), recombinant human IL-2 (Proleukine, Chiron, Amsterdam, 100U/mL), and a tolerogenic cocktail: TGFβ (5 ng/mL), PGE2 (500 nM) and rapamcyin (10 nM), in the presence of ΔCD3-feeder. For plate-bound CD3 stimulation, 100 µL of the anti-CD3 mAb diluted into PBS (Invitrogen) were added to each culture well, placed at 4° C. for 16 h, and then washed twice with PBS. Cells were restimulated every week and harvested for analysis after 21 days of culture.

For specific iTreg generation, cells were stimulated in the tolerogenic medium with autologous immature dendritic cells pre-incubated with OVA or synovial fluid for 24 h. Recombinant human IL-2 was added after 3 days of culture. Cells were restimulated every week with pre-incubated autologous immature dendritic cells. After three stimulations, antigen specific memory T cells (CD45RO+ CD25+) were sorted and either analyzed or either expanded with polyclonal stimulation in the tolerogenic medium, as described previously.

### 4) Flow Cytometry Analysis:

a) mAb labelling. A multicolor immunophenotyping approach was used for the identification and analysis of different lymphocyte subpopulations. Immunophenotypic studies were performed on fresh or frozen samples, using 11 to 18-colour flow cytometry. Four common membrane markers (CD4-FITC (13B8.2) Beckman Coulter, CD45RA-BV650 (HI100) BD Biosciences, CD45RO-APC-eF780 (UCHL1) Invitrogen, CD25-BV785 (M-A251) BD Biosciences, IL-1RI-PE R&D), an intracellular marker (FOXP3-PECF594 (236A/E7), BD Biosciences) and a viability dye were constantly present in all aliquots. Cells were stained for membrane markers (at 4° C. in the dark for 30 min) using cocktails of Ab diluted in PBS containing BSA/NaN3 (0.5% BSA, 0.01% NaN3) (FACS buffer). FOXP3 intracellular staining was performed according to the manufacturer’s instructions. Appropriate isotype control Abs were used for each staining combination. Samples were acquired on BD LSR-Fortessa flow cytometer using FACSDiva software (Beckton Dickinson). Flow data were analysed using FlowJo software (FlowJo, LLC).

b) CFSE staining. nTreg cells or Tconvs were stained with 1 µM CFSE (Cell Trace cell proliferation kit; Molecular Probes/Invitrogen) in PBS for 8 min at 37° C. at a concentration of 1 × 10⁶ cells/mL. The labelling was stopped by washing the cells twice with RPMI-1640 culture medium containing 10% FBS. The cells were then re-suspended at the desired concentration and subsequently used for proliferation assays.

### 5) Functional Assays.

a) Polyclonal nTreg cell-contact mediated suppression. CFSE-labelled Tconvs (4 × 10⁴/well), used as responder cells, were cultured with ΔCD3-feeder (4 × 10⁴/well) in the presence or absence of defined amounts of nTreg cells or iTreg (0.4 × 10⁴ to 4 × 10⁴ cells/well) for 4-5 days. Cultures were performed in round bottom wells coated with 0.2 µg/mL anti-CD3 mAb in 200 µL of complete medium. Varying concentrations of soluble anti-CD28 mAb were added when indicated. Results are expressed either as the percentage of proliferating CFSE low T cells or as a percentage of suppression calculated as follows: (100 × [(percentage of Tconv CFSE low cells -- percentage of Tconv CFSE low in coculture with nTreg cells)/percentage of Tconv CSFE low cells]).

b) Ag-specific HLA-DR-restricted nTreg suppressive assay. Pre-activated CFSE-labelled Tconv cells (4 × 10⁴/well) with soluble anti-CD3 (4 µg/mL) and anti-CD28 (4 µg/mL) were stimulated with autologous iDC (10⁵ cells/well of each cell type) in the presence or absence of defined amounts of autologous nTreg or iTreg (0.4 × 10⁴ to 4 × 10⁴ cells/well) for 4-5 days.

6) Cytokines quantification. IL-17 levels in cell culture supernatants (SN) were determined by luminex technology.

7) qRT-PCR. Total RNA was isolated from nTreg or iTreg conserved in RLT buffer, using miRNeasy Micro Kit (Quiagen, Courtaboeuf, France). cDNA was synthesized from total RNA using a reverse transcription kit PrimerScriptTM 1^{st} strand cDNA Synthesis (Takara Bio Europe S.A.S., Saint-Germain en Laye, France). RNA quantitation was performed with a Nanodrop instrument (Thermo Fisher Scientific, Courtaboeur, France). The expression levels of IL-1RI was tested by real-time quantitative PCR. Real-time quantitative PCR was carried out using the kit Power SYBR Green PCR Master Mix (Applied Biosystems, Foster City, California, USA). A LightCycler 480 II thermocycler (Roche Applied Science, Meylan, France) was used. PCR conditions were 95° C. for 5 min, followed by 45 cycles of 95° C. for 15 s and 60° C. for 1 min. At the end of the amplification reaction, a melting curve analysis was performed to confirm the specificity as well as the integrity of the PCR product by the presence of a single peak. Absence of cross-contamination and primer dimers was verified on a blank water control. The geometric means of three reference genes (YWHAZ, TOP1 and ATP5B) was used for normalization. The relative expression levels of mRNA were determined using the ΔΔCt formula; fold changes were calculated as 2-ΔΔCt. Only means of duplicates with a CV of <15% were analysed.

8) Bisulfite pyrosequencing. Primer Design: All primers used in this study are listed and were purchased from Eurofin Genomics. Reverse PCR primers were biotinylated for downstream pyrosequencing experiments. For FOXP3 upstream enhancer 1 and proximal promoter regions, the pyrosequencing primers were designed using the SNP Primer design software (Qiagen). Bisulfite conversion: Bisulfite conversion of DNA was performed on DNA from 5,000-100,000 fixed cell using the EZ DNA Methylation -DirectTM Kit (Zymo Research) according to the manufacturer’s instruction in an elution volume of 12-20 µL.

PCR amplification: For each region, PCR reactions were performed using 1 µL of bisulfite treated DNA as template in a 20 µL PCR mix including 200 nM of each primer, 1x HotStar Taq DNA polymerase Buffer, 1.6 mM of additional MgCl2, 200 µM of each dNTPs, and 2 U of HotStar Taq DNA polymerase. The reaction was performed in a Mastercyler Pro S (Eppendorf) and the cycling conditions included an initial denaturation step performed for 10 min at 95° C., followed by 50 cycles of 30 sec denaturation at 95° C., 30 sec annealing at Ta and 30 sec elongation at 72° C. The final step included 5 min elongation at 72° C. The optimal primer annealing temperatures were determined for each assay using the same PCR and cycling conditions except for the annealing step performed using a gradient temperature program ranging from 50° C. to 70° C., followed by the analysis of 5 µl of the PCR reaction by electrophoresis on a 2% agarose gel.

DNA methylation analysis by pyrosequencing: 10 µL of PCR product were supplemented with 2 µL of Sepharose beads, 40 µL of binding buffer (10 mM Tris-HCl, 2 M NaCl, 1 mM EDTA, 0.1% Tween 20; pH 7.6) and 28 µL of water and incubated under constant mixing (1400 rpm) for 10 min at room temperature. PCR products were then purified and rendered single-stranded using the PyroMark Q96 Vacuum Workstation (Qiagen) after three successive baths of 70 % ethanol, 0.2 M NaOH denaturing solution and 1x washing buffer (10 mM Tris-acetate; pH 7.6). Final elution was performed in a pyrosequencing plate (PyroMark Q96 Plate Low, Qiagen) including 4 pmol of the pyrosequencing primer and 12 µL of annealing buffer (20 mM Tris-acetate, 2 mM Mg-acetate; pH 7.6). DNA methylation analysis was performed using PyroMark Gold SQA Q96 Kit (Qiagen) on a PyroMark Q96 MD (Qiagen) and analyzed with PyroMark CpG software (Qiagen).

9) Immunofluorescence. CD4+ CD25high live cells were sorted from CD31d Foxp3 Treg or naive nTreg after 21 days of culture in tolerogenic medium and 50.000 were cytospinned on slides. Cells were stained using the Image IT Fixation/Permeabilization kit (Molecular Probes, Eugene, Oregon, USA). The following antibodies were used for staining: biotinylated anti-Foxp3 (1:100) and anti-biotin AF594-labeled antibody as a secondary antibody (1:100). Cells were incubated in a medium with 4′6-diamidino-2-phenylindole (DAPI, Vector Laboratories) to trace cell nuclei. Slides were evaluated in a LSM 780 confocal microscope with a 63X NA 1,4 oil immersion objective.

10) Statistical analysis. Difference between groups was assessed using Student’s T-Test. Error bars on graphs represent either s.e.m. or interquartile range. Statistical analysis was performed using GraphPad Prism. P values under or equal to 0.05 were considered as statistically significant. In the figures, P values are displayed according to the following representation: * P< 0.05, ** P<0.005, *** P<0.001.

### Results

### Ex Vivo Generation of CD31d-Foxp3 Treg Cells by Functional Reprogrammation of Activated TH0 Cells

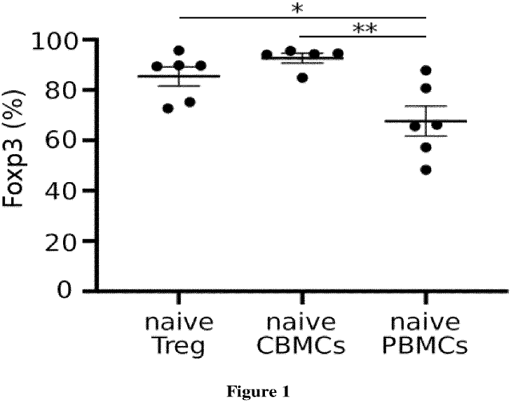

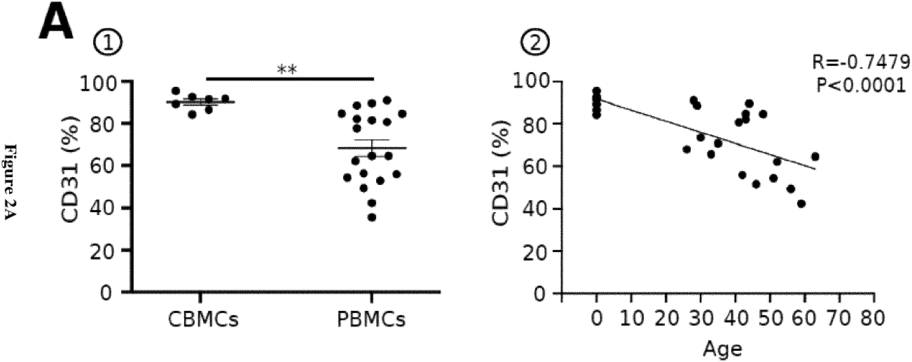

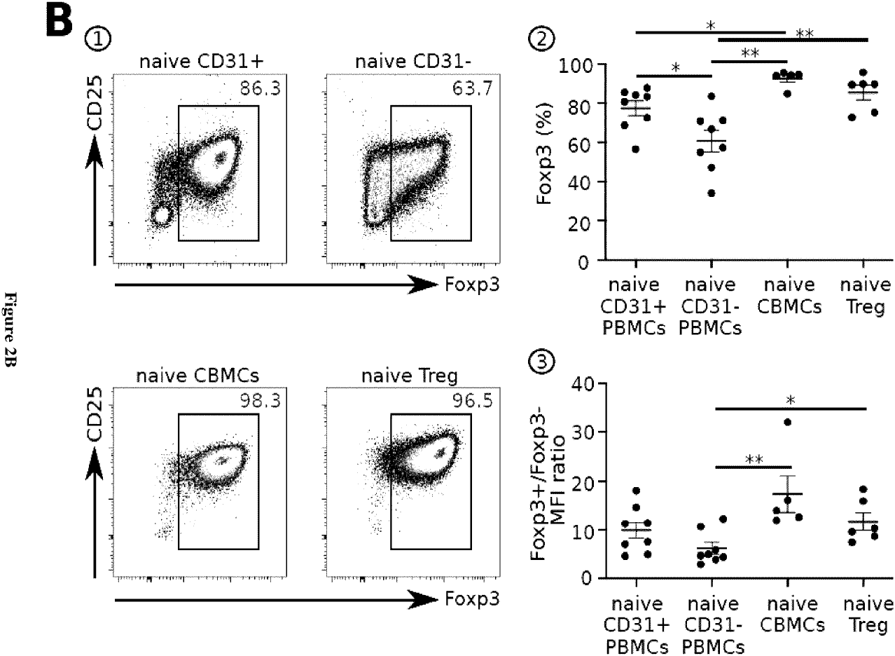

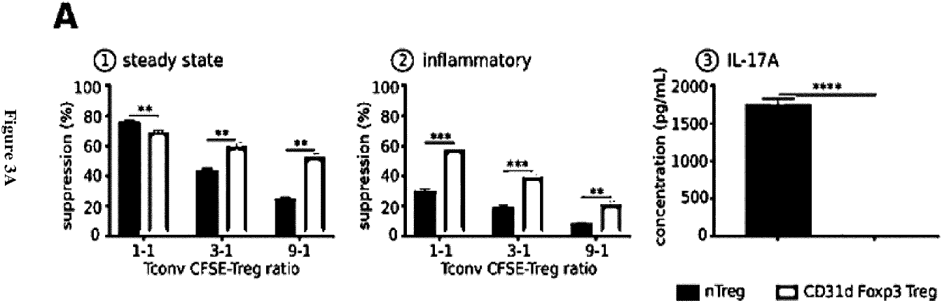

In the prospect of using autologous cells for nTreg adoptive transfer, we investigated whether naïve CD4+ T cells isolated from PBMCs could be functionally reprogrammed (transdetermined) to Foxp3 regulatory T cells under a tolerogenic microenvironment, as can be naïve CD4+ T cells isolated from cord blood (Schiavon et all). We purified naïve human CD4+ CD25- CD127+ CD45RA+ T cells by FACS from PBMCs and CBMCs. These cells will be referred to as PBMCS and CBMCs naïve CD4+ T cells, respectively and ex vivo naïve nTreg cells isolated from PBMCs were used as positive control. FIG. 1 shows that the induction of Foxp3 in PBMCs naïve CD4+ cells is less effective than that in CBMCs naive CD4+ T cells. Upon polyclonal stimulation of these cells with anti-CD3/anti-CD28, IL-2, TGFb, PGE2 and RAPA, CBMCs naïve CD4+ T cells expressed Foxp3 (92.64 +- 4.363). In contrast, only 67.63 % +/- 14.6 of the PBMCs naïve CD4+ T cells could be induced to express foxp3. Exploring the phenotypic difference between both naïve CD4+ T cells, we found that the CD31 marker, characterizing recent thymic emigrants, was significantly less express in PBMcs than in CBMCs naïve CD4+ T cells (68.33% +/- 17.36 versus 90.23% +/- 3.83) (FIG. 2A1). Interestingly the CD31 marker in naïve CD4+ TH0 is correlated to age (FIG. 2A2). These data prompted us to compare the susceptibility of naïve CD31 + from PBMCs to express the foxp3 transcript. We found that CD31+ subset exhibits a higher frequency of Foxp3 expression than CD31- subset. This frequency was similar to the one exhibited by both CBMCs naïve cells and naïve nTreg cells (FIGS. 2B1, B2, B3).

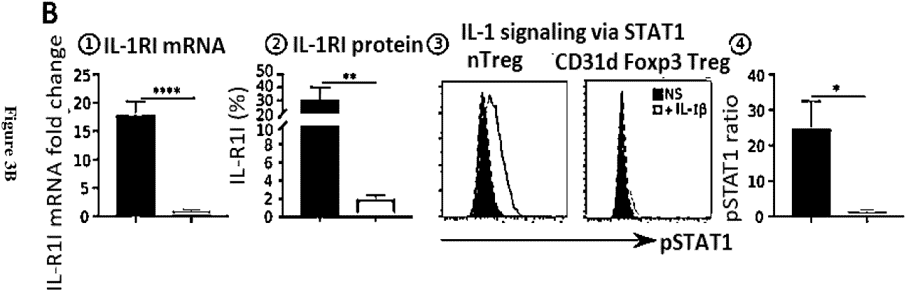

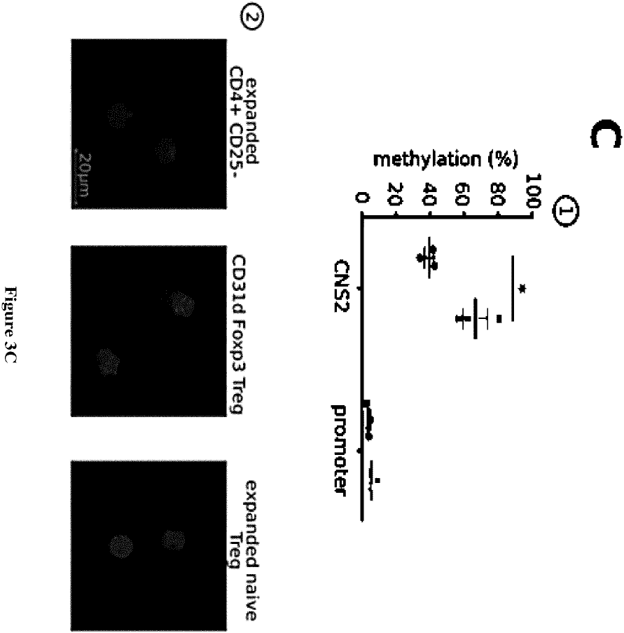

Furthermore we show that these ex vivo generated CD31d-Foxp3 Treg cells display a similar suppressive activity as fresh nTreg when using the standard polyclonal cell-cell contact Treg suppression assay (FIG. 3A1). By contrast, when the functional suppressive assay was performed in presence of a highly-inflammatory conditioned medium containing IL-2, IL-1β, IL-6, IL-21 and IL-23 cytokines, while under these culture condition of stimulation, fresh nTreg loose their suppressive capacity, ex vivo generated CD31d-Treg still maintain their suppressive activity (FIG. 3A2). The maintenance of their suppressive capacity in a high inflammatory context could be ascribed to the fact that they produce background levels of IL-17 when stimulated through CD3 and CD28 in the presence of IMDM medium containing the above inflammatory cytokines compared to fresh nTreg (FIG. 3A3).

Exploring which phenotypic or epigenetic characteristics could explain this different behaviour, we found that transdetermined CD31d Foxp3+ T cells exhibit lower levels of IL-1R1 mRNA (FIG. 3B1) and protein expression (FIG. 3B2) associated with a decrease STAT1 activation following IL-1β stimulation (FIG. 3B3-B4) compared to naive Treg expanded in the same conditions. As to the epigenetic markers, as expected, while CD31d-Foxp3 Treg cells exhibit a higher methylation levels of the Foxp3 CNS2 compared to the expanded naive nTreg cells, both cells revealed comparable levels of Foxp3 promoter methylation (FIG. 3C1). Interestingly CD31d-Foxp3 Treg cells have similar Foxp3 nuclear localization than expanded naive nTreg cells (FIG. 3C2).

### Antigen Specific CD31d-Foxp3 Treg Cells Generation and Expansion.

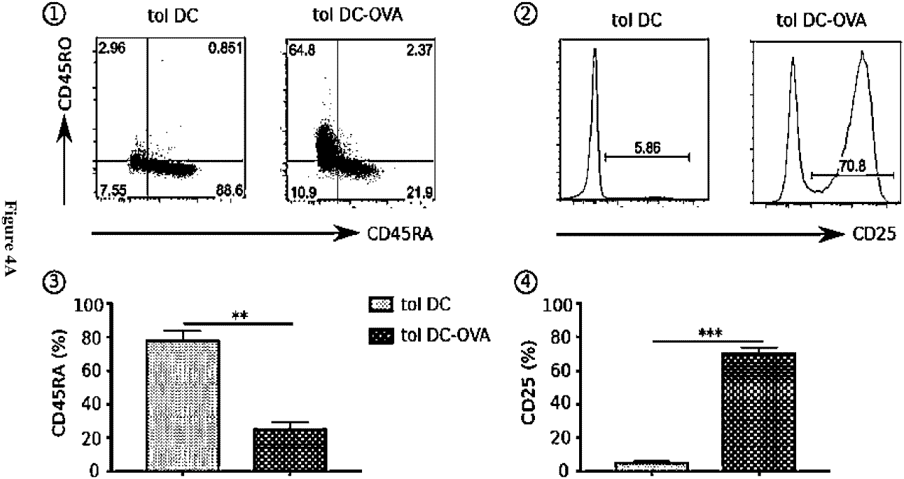

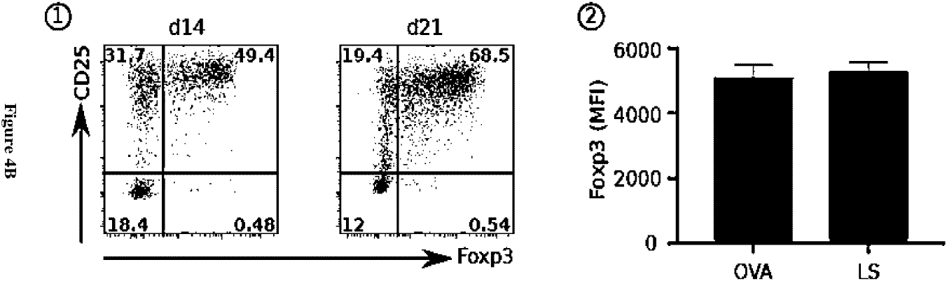

Clinical transfer of polyclonal Treg cells has been shown to be safe in patients as treatment for GVHD and T1D (10,12,13). However, due to their TCR polyclonality, a large number of T cells (in the 10⁹-10¹⁰ range) need to be administered, thus representing a limitation to nTreg based adoptive therapy. In order to overcome this drawback, we set up a method to generate and expand Ag-specific Treg exhibiting highly potent suppressive activity whichever culture condition of stimulation. This protocol consists of generating antigen-specific CD31d-Treg cells by stimulating naïve CD31+ T cells with antigen-pulsed tolerogenic DC (tolDC) in presence of the Treg polarizing medium comprising the combination of IL-2, TGFβ, PGE2 and rapamycin. Briefly, after 3 stimulations under the same conditions, CD4+ T cells that still retained the naive phenotype CD45RA+RO- were removed from the culture and the antigen specific memory T-cells were further expanded with periodic stimulation with anti-CD3 Ab and anti-CD28 Ab in the presence of the same tolerogenic medium as described in Mat and Methods. FIGS. 4A1, A2, A3, A4 shows that OVA-pulsed autologous tolDCs, in presence of the Treg polarizing medium are able to specifically stimulate naive CD31+ T cells, (loss of CD45RA marker and increase expression of CD25), while non-pulsed autologous tolDCs, in presence of the same polarizing medium, were unable to stimulate them (persistence of CD45RA marker and absence of CD25 expression). FIG. 4B1-B2 show also that naïve CD31+ T cells, when specifically activated with OVA-pulsed autologous tolDCs for 21 days are able to express high level and intensity of Foxp3. Also loading tolDC with inflammatory tissue effusion harboring pathogenic Ag as yet unidentified, such as the synovial liquid (LS) from patients suffering of rheumatoid arthritis enabled us to generate and expand tissue Ag specifc Foxp3 Treg cells (FIG. 4B2). In addition, the 21-day-generated-OVA-specific CD31 derived T cells exhibit a higher suppressive activity when using the autologous mixed lymphocytes suppressive assay, whichever the inflammatory context, as compared to fresh nTreg cells (FIG. 4C1-C2). Furthermore, these ova-specific CD31d-Foxp3 Treg cells produce background levels of IL-17 when stimulated through CD3 and CD28 in the presence of IMDM medium containing IL-2, IL-1β, IL-6, IL-21 and IL-23 cytokines compared to fresh nTreg cells (FIG. 4C3). Finally, when the antigen specific (OVA/LS) CD4+ CD25+ CD45RA- T cells are isolated from the autologous mixed lymphocyte culture and polyclonally expanded 21 days more in the tolerogenic medium, these cells still maintained high level of Foxp3 expression and their suppressive function whichever the inflammatory context. When starting with 1 × 10⁶ TH0 cells, this protocol can yield an average of 50 × 10⁶ Ag specific CD31d- Foxp3 Treg cells.

### Conclusion

nTreg based adoptive therapy represents a promising medication to autoimmune pathologies, such as type I diabetes, and in prevention of GVHD, or of allogeneic transplant rejection in solid organ transplantation. However, nTreg cell preparations currently administered in patients exhibit intrinsic characteristics, which per se represent a source of clinical complications and limitations. Indeed, they are most often neither autologous nor Ag specific. Furthermore, they may exert a TH-17 like activity under a pro-inflammatory tissue stromal context (Le Buanec /VS PNAS). The present study focuses on the CD31d Foxp3 Treg cells. The structural and functional characteristics of these Treg cells are distinct from those of the currently administered Treg cells including fresh or expanded nTreg cells of thymic origin, ex vivo induced nTreg (ref) and car Treg cells (ref). They are optimal for the use of CD31d Foxp3 Treg cells in Treg-based adoptive therapies, considering that 1) these cells can be generated from autologous cells, 2) they can be expanded under appropriate conditioned medium for over 21 days remaining Ag specific, 3) following Ag specific stimulation their number could be administered at much lower doses than ex vivo-expanded polyclonally stimulated Treg cells. Given that the number of antigen specific CD31d Foxp3 Treg cells generated from only 1 million blood TH0 could exceed 8-10 million cells, the number of these cells required for adoptive therapy should not represent a limitation, as compared to the polyclonally expanded cells administered in current trials to treat both auto and allo-immune disease range from 3 to 300 million cells (Table 1). In this line it is noteworthy to consider that following Ag specific stimulation with auto-immune tissue extracts, as described in this study with synovial liquid originated from rheumatoid arthritis patients, CD31d Foxp3 Treg cells could control in auto-immune pathologies the different pathogenic autoreactive clones induced by the distinct auto-antigens even when unidentified. 4) Most importantly, given that these cells do not released IL-17, their adoptive transfer for therapeutic purpose does not entail any risk of inflammatory complication.

### EXAMPLE 2

### Material and Method

1) Human blood samples. Peripheral Blood samples were obtained from healthy donors through Etablissement Français du Sang (EFS, Paris, France) and from treated or untreated patients with Systemic Sclerosis through Discovery Life Science, Alabama, United States or with Systemic Lupus Erythematosus through Hôpital Saint Louis, Paris.

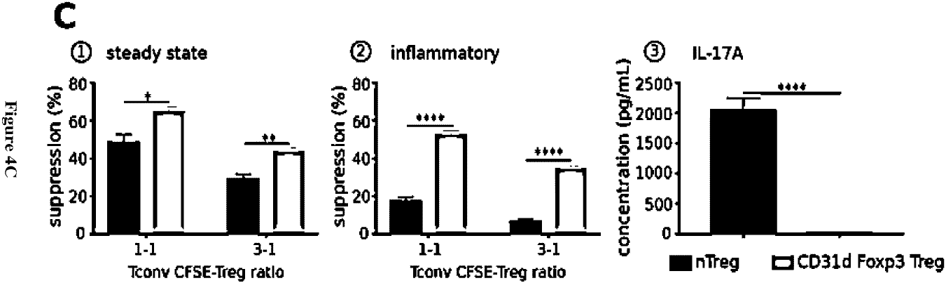

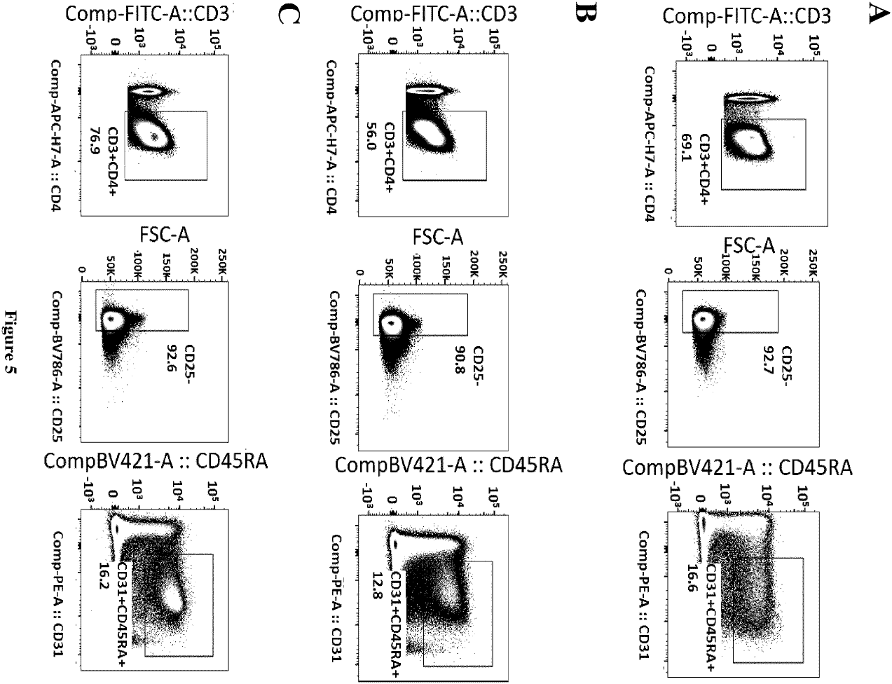

2) Cell purification. Peripheral blood mononuclear cells (PBMCs) were isolated by density gradient centrifugation on Ficoll-Hypaque (Pharmacia, St Quentin en Yvelines, France). PBMCs were stored frozen in liquid nitrogen. CD4+ T cell subsets and T cell-depleted accessory cells (ΔCD3 cells) were isolated from frozen PBMCs. All CD4+ T cells were positively selected with a CD4+ T cell isolation kit (Miltenyi Biotec, Bergisch-Gladbach, Germany), yielding CD4+ T cell populations at a purity of 96-99%. Subsequently, selected CD4+ T cells were labelled with anti-CD25 (B1.49.9)-PC5.5 (Beckman Coulter), anti-CD45RA (REA562)-FITC (Miltenyi) and anti-CD31 (WM59)-PE (Biolegend) before being sorted into naive CD31+ naïve Tcells (CD4+ CD25neg/ dim CD45RA+CD31+/-) subpopulations using a FACSARIAIII Cell Sorter (Becton Dickinson, Le Pont Claix, France) (FIGS. 5A, 5B, 5C, Table 2). Postsort analysis confirmed that the purity for each cell type was routinely greater than 80-90%.

3) Culture. For polyclonal iTreg generation, cells were stimulated with plate-bound anti-human CD3 (OKT3) mAb (eBioscience, San Diego, CA) at the concentration of 1 µg/mL, soluble anti-human CD28 (CD28.2) mAb (Becton Dickinson, 2 µg/mL), recombinant human IL-2 (Proleukine, Chiron, Amsterdam, 100U/mL), and a tolerogenic cocktail: TGFβ (5 ng/mL), PGE2 (500 nM) and rapamcyin (10 nM), in the presence of ΔCD3-feeder. For plate-bound CD3 stimulation, 100 µL of the anti-CD3 mAb diluted into PBS (Invitrogen) were added to each culture well, placed at 4° C. for 16 h, and then washed twice with PBS. Cells were restimulated at Day 6 and harvested for analysis at Day 12 of culture.

### 4) Flow Cytometry

a) Phenotypic analysis of unstimulated PBMCs. PBMCs were stained with 2 different antibody panels (Table 3) to evaluate CD31 expression in naïve CD4+ T cells gated either as CD3+ CD4+ CD45RA+ CD25-(Table 3 Panel 1) or as CD3+ CD4+ CD45RA+ CCR7+ (Table 3 Panel 2).

b) Phenotypic analysis of naïve CD4+ T cells stimulated under tolerogenic conditions. CD4+ T cells were stained with the antibody panel described in Table 3 Panel 3.

### Results

### Patients With Systemic Sclerosis and Systemic Lupus Erythematosus Exhibit Naïve CD4+ T Cells Expressing CD31

We wanted to see if patients with autoimmune diseases could present naive CD4+ T cells expressing CD31, in order to generate and expand in vitro stable regulatory T cells regardless of their microenvironment. As shown in Table 4A and Table 4B, patients with Systemic Lupus Erythematosus or Systemic Sclerosis expressed similar level of CD31 in naïve CD4+ T cells as Healthy Donors.

### CD4+ Expressing CD25 and FOXP3 Can Be Generated in Vitro From Naive CD4+ CD31+ T Cells Isolated From Patients with Systemic Sclerosis

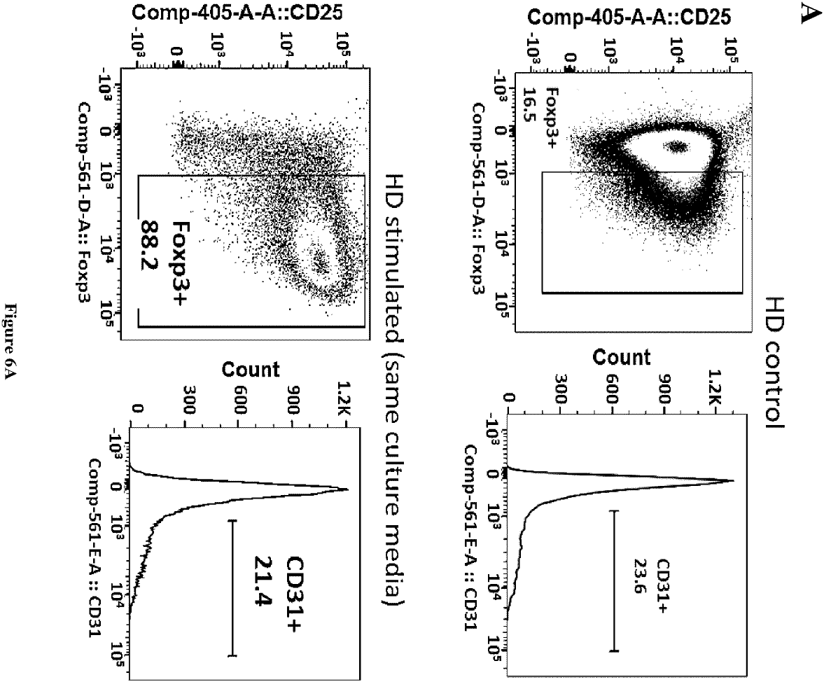

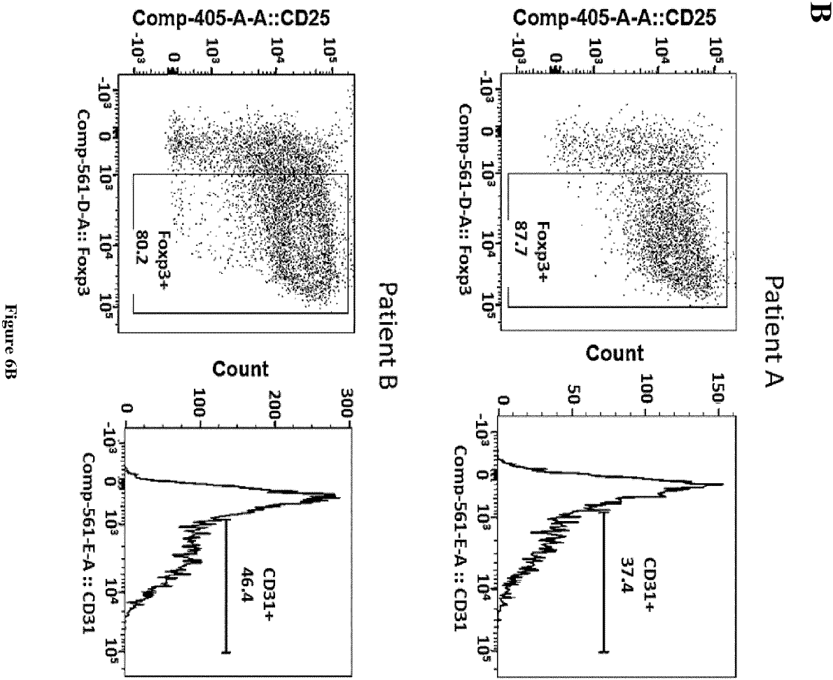

As a result of the tolerogenic medium used to transduce the expression of CD25 and Foxp3 in the population of interest (CD4+ CD25neg/ dimCD45RA+CD31+) during 12 days, we analyzed the expression of Foxp3 and CD25 which are marker of regulatory T-cells (Treg) (Table 5). There was no significant change between the Healthy Donor and the 2 Systemic Sclerosis patients with a population between 82.4% (Patient A) and 69.1 % (Patient B) expressing Foxp3 compared to control HD Stimulated (91.2%) with high level of CD25. In contrary unstimulated Healthy Donor HD do express only very few Foxp3 with 4.7 % (FIGS. 6A, 6B).

Those experiments show the effect of i) cell sorting of the CD4 naïve T-cell CD31+ subpopulation and ii) the tolerogenic medium to transduce the expression of CD25/Foxp3 which characterizes regulatory T-cells (Treg).

We also looked at the expression of CD31 in the “final” population (i.e. after the 12 days of harvesting with the tolerogenic medium): about 37 to 47% of cells do express CD31, which is not the objective of this population of interest. We only want to have Foxp3/CD25 cells indifferently of the expression of CD31, unlike some previous publications.

It confirms the previous results with diseased PBMC from Systemic Sclerosis which is a major auto-immune disease with unbalanced Treg/Th17 in favor of Th17.

### Tables

TABLE 1

Published results on Treg adoptive transfer in human disease.

| | | | | |
|---|---|---|---|---|
| Publication | Diseases | Cells | Dose | Expansion |
| Trzonkowski and al, 2009 | GVHD | Polyclonal expanded Treg | 1 × 10⁵ or 3 × 10⁶ Treg / kg | αCD3 + αCD28 + IL-2 |
| Brunstein and al, 2011 | GVHD | Polyclonal expanded UCB Treg | 0.1-30 × 10⁵ Treg /kg | αCD3 + αCD28 + IL-2 |
| Di Ianni and al, 2011 | GVHD | Fresh polyclonal Treg | 2-4 × 10⁶ Treg / kg | No expansion |
| Marek-Trzonkowska and al, 2012 | T1DM | Polyclonal expanded Treg | 10-20 × 10⁶ Treg /kg | αCD3 + αCD28 + IL-2 |
| Desreumaux and al, 2012 | Crohn’s disease | OVA-specific Tr1 | 1 × 10⁶ - 1 × 10⁹ Treg | αCD3 + IL-2 + OVA then selection of OVA-specific IL-10-producing cells |
| Bachetta and al, 2014 | GVHD | IL-10 DLI | 1 × 10⁵ - 3 × 10⁶ T cells / kg | Donor T cells pretreated with IL-10 |
| Martelli and al, 2014 | GVHD | Fresh polyclonal Treg | 2.5 × 10⁶ Treg / kg | No expansion |
| Bluestone and al, 2015 | T1DM | Polyclonal expanded Treg | 0.05-26 × 10⁸ Treg | αCD3 + αCD28 + IL-2 |
| Theil and al, 2015 | GVHD | Polyclonal expanded Treg | 2.4 × 10⁶ Treg / kg | αCD3 + αCD28 + IL-2 + rapamcyin |
| Todo and al, 2016 | Liver transplant ation | Regulatory lymphocytes | 23.30 × 10⁶ T cells /kg | Allo-reactive regulatory lymphocytes |
| Chandran and al, 2017 | Kidney transplant ation | Polyclonal expanded Treg | 320 × 10⁶ Treg | αCD3 + αCD28 + IL-2 |
| Mathew and al, 2018 | Kidney transplant ation | Polyclonal expanded Treg | 0.5-5 × 10⁹ Treg | αCD3 + αCD28 + IL-2 + TGFβ + rapamcyin |
| Kellner and al, 2018 | GVHD | Fucosylated polyclonal expanded UCB Treg | 1 × 10⁶ Treg / kg | αCD3 + αCD28 + IL-2 |
| Thonhoff and al, 2018 | Amyotrop hic lateral sclerosis | Autologous polyclonal expanded Treg | 1 × 10⁶ Treg / kg | αCD3 + αCD28 + IL-2 + rapamcyin |

Abbreviations: GVHD, graft-versus-host disease; T1DM, type I diabetes mellitus

TABLE 2

Sorting iTreg: Percentage CD4 naïve CD31+

| Sample | % CD45RA+CD31+ |
|---|---|
| HD H61 | 16.6 |
| Patient A | 12.8 |
| Patient B | 16.2 |

Percentage (%) of CD45RA+ CD31+ from Naïve CD4 CD25- cells.

TABLE 3:

antibody panels for flow cytometry analysis

| | |
|---|---|
| Markers | Fluorochrome |
| CD3 | FITC |
| Viab | V506 |
| CD4 | APC-H7 |
| CD25 | BV786 |
| CD45RA | BV421 |
| CD31 | PE |

Panel 1: ex vivo phenotypic analysis

| | |
|---|---|
| Markers | Fluorochrome |
| CD3 | FITC |
| Viab | V506 |
| CD4 | APC-H7 |
| CCR7 | BV786 |
| CD45RA | BV421 |
| CD31 | PE |

Panel 2: ex vivo phenotypic analysis

| | |
|---|---|
| Markers | Fluorochrome |
| CD3 | BUV737 |
| Viab | V506 |
| CD₄ | BUV 395 |
| CD25 | BV786 |
| CD45RA | BV650 |
| CD31 | PE |
| Foxp3 | PeCF594 |

Panel 3: phenotypic analysis after in vitro stimulation in presence of tolerogenic condition

TABLE 4A:

Frequencies of CD31+ T cells within the CD3+ CD4+ naïve compartment from patients with Systemic Lupus Erythematosus (SLE) under treatment.

| Gating Strategy | Healthy Donor 1 | | | Healthy Donor 2 | | |
|---|---|---|---|---|---|---|
| | Event Count | % of Total | % of parent | Event Count | % of Total | % of parent |
| Ungated | 953200 | | | 588512 | | |
| Lymphocytes | 523749 | 54.95 | 54.95 | 343002 | 58.28 | 58.28 |
| CD3+ | 366149 | 38.41 | 69.91 | 241035 | 40.96 | 70.27 |
| CD3+ CD4+ | 224810 | 23.58 | 61.4 | 108586 | 18.45 | 45.05 |
| Naive CD3+ CD4+ | 118631 | 12.45 | 52.77 | 32315 | 5.49 | 29.76 |
| Naive CD3+ CD4+ CD31+ | 88427 | 9.28 | 74.54 | 24638 | 4.19 | 76.24 |
| Gating Strategy | Patient 1 with Systemic Lupus Erythematosus under treatment | | | Patient 2 with Systemic Lupus Erythematosus under treatment | | |
| | Event Count | % of Total | % of parent | Event Count | % of Total | % of parent |
| Ungated | 788256 | | | 1204760 | | |
| Lymphocytes | 671068 | 85.13 | 85.13 | 1003262 | 83.27 | 83.27 |
| CD3+ | 439875 | 55.8 | 65.55 | 860753 | 71.45 | 85.8 |
| CD3+ CD4+ | 305775 | 38.79 | 69.51 | 591459 | 49.09 | 68.71 |
| Naive CD3+ CD4+ | 243651 | 30.91 | 79.68 | 303375 | 25.18 | 51.29 |
| Naive CD3+ CD4+ CD31+ | 181368 | 23.01 | 74.44 | 203841 | 16.92 | 67.19 |

Frequencies of CD31 + T cells within the CD3+ CD4 + naive compartment. (A) PBMCsfrom 2 Healthy Donors and 2 SLE patients under treatment were analyzed by flow cytometry with a panel using antibodies anti CD3, CD4, CD45RA, CCR7, and CD31 to identify naive (CD45RA+, CCR7+) CD4+ expressing CD31 as a marker of recent thymic emigrants.

TABLE 4B:

Frequencies of CD31 + T cells within the CD3+ CD4 + naive compartment from patients with systemic sclerosis.

| | Healthy Donor 3 | | | Patient 1 with systemic sclerosis under treatment | | |
|---|---|---|---|---|---|---|
| Gating Strategy | Event Count | % of Total | % of parent | Event Count | % of Total | % of parent |
| Ungated | 1982394 | | | 1146256 | | |
| Lymphocytes | 440014 | 22.2 | 22.2 | 474505 | 41.4 | 41.4 |
| CD3+ | 240586 | 12.1 | 55.3 | 297568 | 26 | 63 |
| CD3+ CD4+ | 166152 | 8.38 | 69.1 | 250006 | 21.8 | 84 |
| Naive CD3+ CD4+ | 76860 | 3.88 | 46.3 | 175216 | 15.3 | 70.1 |
| Naive CD3+ CD4+ CD31+ | 26923 | 1.36 | 35 | 94770 | 8.27 | 54.1 |
| | Patient 2 with systemic sclerosis under treatment | | | Patient 3 with systemic sclerosis without treatment | | |
| Gating Strategy | Event Count | % of Total | % of parent | Event Count | % of Total | % of parent |
| Ungated | 3022731 | | | | | |
| Lymphocytes | 1740055 | 57.6 | 57.6 | 1025239 | 46.8 | 46.8 |
| CD3+ | 788848 | 26.4 | 45.9 | 313365 | 14.3 | 31.3 |
| CD3+ CD4+ | 441398 | 14.6 | 56 | 240931 | 11 | 76.9 |
| Naive CD3+ CD4+ | 184512 | 6.1 | 41.8 | 84058 | 3.84 | 34.9 |
| Naive CD3+ CD4+ CD31+ | 55935 | 1.85 | 30.3 | 39463 | 1.8 | 46.9 |
| Healthy Donor 3 | anti-CD3 / anti CD28 Ab | IL-2 | 4.7 | 96.2 | 11070 | |
| | anti-CD3 / anti CD28 Ab | IL-2 + tolerogenic coktail | 91.2 | 96.3 | 29236 | |
| Patient 1 with systemic sclerosis under treatment | anti-CD3 / anti CD28 Ab | IL-2 + tolerogenic coktail | 82.4 | 96.3 | 22502 | |
| Patient 2 with systemic sclerosis under treatment | anti-CD3 / anti CD28 Ab | IL-2 + tolerogenic coktail | 69.1 | 95.6 | 28546 | |

(A) PBMCsfrom 1 Healthy Donor and from 3 patients with sytemic sclerosis with or without treatment were analyzed by flow cytometry with a panel using antibodies anti CD3, CD4, CD45RA, CD25, and CD31 to identify naive (CD45RA+, CD25-) CD4+ expressing CD31 as a marker of recent thymic emigrants.

### REFERENCES

Throughout this application, various references describe the state of the art to which this invention pertains. The disclosures of these references are hereby incorporated by reference into the present disclosure.

1. Cao D, et al. (2003) Isolation and functional characterization of regulatory CD25brightCD4+ T cells from the target organ of patients with rheumatoid arthritis. Eur J Immunol 33(1):215-223.

2. Miyara M, et al. (2005) Global natural regulatory T cell depletion in active systemic lupus erythematosus. J Immunol (Baltimore, Md 1950) 175(12):8392-8400.

3. Liu B, et al. (2007) Abnormality of CD4(+)CD25(+) regulatory T cells in idiopathic thrombocytopenic purpura. Eur J Haematol 78(2):139-143.

4. Salama AD, Najafian N, Clarkson MR, Harmon WE, Sayegh MH (2003) Regulatory CD25+ T cells in human kidney transplant recipients. J Am Soc Nephrol 14(6):1643-51.

5. Zorn E, et al. (2005) Reduced frequency of FOXP3+ CD4+CD25+ regulatory T cells in patients with chronic graft-versus-host disease. Blood 106(8):2903-11.

6. Matsuoka K, et al. (2010) Altered regulatory T cell homeostasis in patients with CD4+ lymphopenia following allogeneic hematopoietic stem cell transplantation. J Clin Invest 120(5):1479-93.

7. Tang Q, Lee K (2012) Regulatory T-cell therapy for transplantation: how many cells do we need? Curr Opin Organ Transplant 17(4):349-54.

8. Putnam A, Bluestone J, Gitelman S, Herold K Results Following Completion of Phase I Safety Trial Using CD4+CD127lo/-CD25+ Polyclonal Treg cells for the Treatment of Recent Onset Type 1 Diabetes.

9. Marek-Trzonkowska N, et al. (2012) Administration of CD4+CD25highCD127-regulatory T cells preserves β-cell function in type 1 diabetes in children. Diabetes Care 35(9):1817-20.

10. Bluestone JA, et al. (2015) Type 1 diabetes immunotherapy using polyclonal regulatory T cells. Sci Transl Med 7(315):315ra189.

11. Trzonkowski P, et al. (2009) First-in-man clinical results of the treatment of patients with graft versus host disease with human ex vivo expanded CD4+CD25+CD127- T regulatory cells. Clin Immunol 133(1):22-26.

12. Di Ianni M, et al. (2011) Treg cells prevent GVHD and promote immune reconstitution in HLA-haploidentical transplantation. Blood 117(14):3921-3928.

13. Brunstein CG, et al. (2011) Infusion of ex vivo expanded T regulatory cells in adults transplanted with umbilical cord blood: safety profile and detection kinetics. Blood 117(3):1061-1070.

14. Theil A, et al. (2015) Adoptive transfer of allogeneic regulatory T cells into patients with chronic graft-versus-host disease. Cytotherapy 17(4):473-486.

15. Bacchetta R, et al. (2014) Immunological outcome in haploidentical-HSC transplanted patients treated with IL-10-anergized donor T Cells. Front Immunol 5(JAN):1-14.

16. Desreumaux P, et al. (2012) Safety and Efficacy of Antigen-Specific Regulatory T-Cell Therapy for Patients With Refractory Crohn’s Disease. Gastroenterology 143(5):1207-1217.e2.

17. Trzonkowski P, et al. (2009) First-in-man clinical results of the treatment of patients with graft versus host disease with human ex vivo expanded CD4+CD25+CD127- T regulatory cells. Clin Immunol 133(1):22-26.

18. Tang Q, et al. (2004) In vitro-expanded antigen-specific regulatory T cells suppress autoimmune diabetes. J Exp Med 199(11):1455-65.

19. Masteller EL, et al. (2005) Expansion of Functional Endogenous Antigen-Specific CD4+CD25+ Regulatory T Cells from Nonobese Diabetic Mice. J Immunol 175(5):3053-3059.

20. Huter EN, Stummvoll GH, DiPaolo RJ, Glass DD, Shevach EM (2008) Cutting edge: antigen-specific TGF beta-induced regulatory T cells suppress Th17-mediated autoimmune disease. J Immunol 181(12):8209-13.

21. Stephens LA, Malpass KH, Anderton SM (2009) Curing CNS autoimmune disease with myelin-reactive Foxp3 + Treg. Eur J Immunol 39(4):1108-1117.

22. Sagoo P, et al. (2011) Human regulatory T cells with alloantigen specificity are more potent inhibitors of alloimmune skin graft damage than polyclonal regulatory T cells. Sci Transl Med 3(83):83ra42.

23. Golshayan D, et al. (2007) In vitro-expanded donor alloantigen - specific CD4+CD25+ regulatory T cells promote experimental transplantation tolerance. Blood 109(2):827-835.

24. Valencia X, et al. (2006) TNF downmodulates the function of human CD4+ CD25 hi T-regulatory cells. Immunobiology 108(1):253-261.

25. Zheng SG, Wang J, Horwitz DA (2008) Cutting Edge: Foxp3+CD4+CD25+ Regulatory T Cells Induced by IL-2 and TGF- Are Resistant to Th17 Conversion by IL-6. J Immunol. doi:10.4049/jimmunol.180.11.7112.

26. Xu L, Kitani A, Fuss I, Strober W (2007) Cutting Edge: Regulatory T Cells Induce CD4+CD25-Foxp3- T Cells or Are Self-Induced to Become Th17 Cells in the Absence of Exogenous TGF- . J Immunol 178(11):6725-6729.

27. Shultz LD, Brehm MA, Bavari S, Greiner DL (2011) Humanized mice as a preclinical tool for infectious disease and biomedical research. Ann N Y Acad Sci 1245:50-4.

1. A population of CD31d-Treg cells having the following phenotype: CD4⁺CD25⁺ CD121a⁻CD127⁻Foxp3⁺.

2. The population of CD3 1d-Treg cells of claim 1 that is a population of CART cells.

3. A method of generating the population of CD31d-Treg cells of claim 1 comprising stimulating naïve CD31+ T cells with antigen-pulsed tolerogenic dendritic cells (tolDC) in the presence of the Treg polarizing medium comprising IL-2, a cAMP activator, a TGFβ pathway activator, and an mTOR inhibitor.

4. The method of claim 3 wherein the tolerogenic DCs express on their surface the major histocompatibility (MHC) class Ia and/or MHC class Ib.

5. The method of claim 3 wherein the tolerogenic DCs are pulsed in the presence of at least one self-peptide antigen, modified self-peptide antigen, over-expressed self-peptide antigen or foreign antigen.

6. The method of claim 3 wherein the cAMP activator is selected from the group consisting of prostaglandin E2 (PGE2), an EP2 or EP4 agonist, a membrane adenine cyclase activator and a metabotropic glutamate receptors agonist.

7. The method of claim 3 wherein the TGFβ pathway activator is selected from the group consisting of TGFβ, bone morphogenetic proteins (BMPs), growth and differentiation factors (GDFs), anti-müllerian hormone (AMH), activin, and nodal.

8. The method of claim 3 wherein the mTOR inhibitor is selected from the group consisting of rapamycin and its analogs ; wortmannin; theophylline; caffeine; epigallocatechin gallate (EGCG); curcumin; resveratrol; genistein; 3, 3-diindolylmethane (DIM); LY294002 (2-(4-morpholinyl)-8-phenyl-4H-1-benzopyran-4-one); PP242; PP30; Torin1; Ku-0063794; WAY-600; WYE-687; WYE-354; and mTOR and PI3K dual-specificity inhibitors.

9. The method of claim 3 wherein the naïve CD31+ T cells are cultured for at least 5 days, at least 6 days, at least 7 days, at least 8 days.

10. The method of claim 3 which further comprises a step of expanding the population of CD31d-Treg cells in the presence of the Treg polarizing medium and a hypomethylating agent.

11. The method of claim 3 which further comprises a step of expanding the population of CD31d-Treg cells in the presence of the Treg polarizing medium and a TCRαβ cell activator.

12. The method of claim 11 wherein the TCR αβ activator is an anti-TCR αβ antibody.

13. The method of claim 10 wherein the population of CD3 1d-Treg cells is expanded in culture for at least 5 days, at least 6 days, at least 7 days, or at least 8 days.

14. A method of treating an autoimmune inflammatory disease in a subject in need thereof comprising administering to the subject a therapeutically effective amount of the population of CD31d-Treg cells of claim 1.

15. A pharmaceutical composition comprising the population of CD31d-Treg cells of claim 1 and at least one pharmaceutically acceptable excipient.

16. (canceled)

17. A method of treating an autoimmune inflammatory disease in a patient in need thereof comprising administering to the patient a therapeutically effective amount of the population of Treg cells of claim 1, wherein the Treg cells are engineered to repress the expression of IL-1R.

18. A method of treating an autoimmune inflammatory disease in a subject in need thereof comprising administering to the patient a therapeutically effective amount of an antibody capable of depleting the population of Treg cells that express the IL-1 receptor.

19. A population of Treg cells engineered to repress the expression of the IL-1 receptor.

20. The population of Treg cells of claim 19 that are also engineered to express a chimeric antigen receptor (CAR).
